SEQUENCE LISTING (48 sequences)

SEQ ID NO: 1 - DNA, 16125 bp, Homo Sapiens
  misc_feature (1)...(16125): n = A,T,C or G
ccaaaaaattgttctcttgggggttggggcgacaagcgggaagggagggcattttgggcaaattggcttattgccacgcaagggctttaacaccttaggttggtgggttcacaggttgcaggcaacccaccatggcacacgtatacctatgtaaccaacctgcaccatcatgtatacctatgtaaccaacctggtacattctgcacacgtatcccaggactttagagtgaaaaaaaaagtggtgtgtagaaaaatcacctgcaatctcagcatagttaacgcttagtacatttcagagagagagggtgacaggaaagggaggatgagagtgggtttaagacacaaggtcatattataaaatcagggcttctggaagtttagtcccaaaaccacacatctcataatcccctgcagtgcttgattaaaatgcaacatccctaaggccacagactcagactctggagaaagatccagaaaactgcccgtttaataaacatttgggcgattcttacggcctctaaagaccaagaaccactgctgcctagagctctgctctcttcattgaacaatacaagaggagtgtgtaggtagacacccaccacttccaacagcttaggagagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcacccccctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggatcatcggacccacctggaaccccacctgtgagtacaaggtgccccaggtggactgggctggggctttgaggccttcagggttggatggccatcttgcgtatttgtgtgggatatgcacacacaggcagcacatgcgcaggtgtgtgggcacctgtgtgtctgtgcaaatgccctgaggtgggaatgagcttggtgtgcatcaggagcgacagccagccagtgtggctgcagcaaaacacacagggaaagaatggagggggcatcaatcactgacaaaattatttatagagctccccctaaaaaaaagaaggtctcttctttcgatagaagaagggagagagggggtttgtccttataaatataagggaggagccgcccctcaaaaaataagggagggaggacccaagaccccgtgggttgtgtgttttccagggggagctcgaaccctttagagggagcgtgggagaaccgctgtattcaggcctctcgagagaaaaggagcggccgcccaaaaaatatccctcccgggcgataagaaatggtggcctctctcaaaaagatgaagaggaagccggagttgtatgtgttgatatttttaaaactccaggtagnnnnnnnnnntgcttcagtaaatttttattgagcgccttctacgagaacacaagaggagcttccattctgaggaggaaacaggcaggaaacaggcagatatcctgtataatttcaagtagtgataagtgctctctagaaatatcaagcaaggtgaggagacacagagcaccggtggcagtggggctctatttccaggttggatggttgggaacatcctttctaaagggaacctggagtgggaaggaaccatgcaggtatctcaggaagagcttcctccaggcaggaagatcagcaggtggaaaggccctggagccaccattcagtaaacatcatttgagcatctctaccagctaggttccattatgggaatgggaatatggtggtggacagggctgcctggtcccttccatacttctcacactagggtggttgagagagcttgggagctaacgaacaagatgggctgagaacactgcctagcccagaggacctgagcttagtgtgtagacattgctgctgttactgcctttgtcgttgtattatttatttatttatttattgatcttaagacagagttttgctcttcttacccaggcttgagtgcaatggcgtgatctcagctcactgcaacctccacctcctgggatcaagcgattctcctgcctcagcctcctgagtagctgggattacaggcacccgcaccacgcctggataatttttttgtatttttagtagagacagggtttcaccatgttggccaggctggtctcgaactcctgaccttaggtgatccacctgcctcgacttcccaaagtgctgggattataggcatgagccactgcgcccagtgattatagaaagttaaaggcacatggcaatgcacacgcctatctacgtcttccctgccaaagcaaagggcagcctctgggctcactttcttgcgtttctacttccaaaaggcagtcagaactggcagggccttggagaccacttcatccacctcctagggtccctatgggagagttgaggtccagagcagggaagggtcctgacaggctctgaccagggcctctgatccctacaaacccccaatcggtgtccctctctaccaggacccaagcccacctgctgcagcccactgcctggccatgaccatcacttacacaagccaagtggctaatgcccgcttaggctccttctcccgcctgctgctgtgctggcggggcagcatctacaagctgctatatggcgagttcctaatcttcctgctctgctactacatcatccgctttatttataggtaaagctggcagggctgggccggggggcctgggaaggatgtggctggggctgggagctgggagctcctgggggcctcccagccagctcagggcccagtgcaccagtccactacaacactaagctgggctcctgaccagctcctgggcactggagctgaggctgcgcgctgggggctgggcagagtaaagaagtcacactgagaggctgctcaagccaggccagcagggttttagccacccttcctccaaccccaggaggacccctggagcccaggctttgtctggccccactctactggcctgttttactgaatcccacacagactcataggcccacatagtacattaaaaaagagagagagagagagagagagagagagatggagtctcactgtgttgtccaggctggtctcgaactcctaggctcaagcaatccccctgccttagcctcccaaggggctgggattacaggtgtgagctactgcacttgaccaaccacatggtactttttttttttttttttttttttgagacagggtttcactccatcacccaggctggagtgcagtgggggcaatcttggctcactgtaacctctgcctcccaggtgcaagcgattctcctgccttagcctcctgagtagctggaattataggcacacaccaccacgcctggctaatttttttttttttctgtatttttagtagagacagggtttcatcatgttggccaggctggtcttgaacccctgacctcaagtgatccacccacctcggcctcccaaagtgctgggattacaggtgtcagccaccatgcacagcccacatggtacattttttaaaattattttttaattaaaatgtttatctaaggccagtagcagtgactcgcgtctgtaatcccagcactttgaggggccaaggtgcggggatcacttgagcctgggagttcagcgtgggcaacatagtgagaccccgtctctaccaaaaatttaaaaaattagctgggagtggtggcatttgcctgtggtcccagctacttgggaagctgaggtgtggggatggctgaagcctgtgaggtcgaggctgcagtgagctatgatcacaccactgcacttcagcctgagtgacaggctatctcaaaagcaaacaaaataatgtttatctaaacggtaaggtataatcacagaatatatgatagcattttaaattgaaaaagcattaatgattacatggattgtaaaatatcaaatacatgaaattcttgtgttcttaataatgctagcaacaaggcacatttggtttttactagggcaccaaggtactttaaaaaaagttagggccagccacaggggctcacacctgtaatcccagcactttgggaggccaaggcaggaggatcacttgagcccaggagtttaggacctgagcaacatagggagatcctgatcttgtctctataaaaaattaaaaaattggctaggccctttggcttacacccgtaatcccagcactttgggaggccgaggcgggtggatcatgaggtcaggagttcaagaccagcctggccaacatagtgaacccaatctctactataaatacaaaaattagccgagtggggtggcacgcacctgtagttccagctactcaggaggatgaggccggagaatcgcttgagcccgggaggcagaggctgcagtgagccgagaccatgccattgcactccagcctaggtgacagagtgagactccgtcttaaaataatattaaaatcttaaaatgatctgggcatggtggcttatgcctgtagtcccacccagctcttcaggaggctgaagcgggaggattgcttcagcccaggaggttgaggctgcagtgagtcatgactgtgccgctgcccttgagcctgggtaacagagcaagaccctatctcaaaacaaacaaacaaacaaacaaacaaacaaaaaccaataaaccaaaaacatttatctaaacaataaaataaaggacagatataatcaccgaatatatgatagcattttaaattgaaaaagcactaatgactacaatggattataaaacatcaaatacataaaattcttaagttcctcctaataccaaatacaaagcacattggtctttggtttttacttgggcaccaatgcatgctgaaaaagagtcgttcattttttagagtagttttaggttcacagcaaaattgagcagaaggtagagttctcatgtgtctctttgctcctccccctgcccccagcctccccactatcaacacccccacactacagtggtagatttattacaatccctgaacccacagtgacacatcactatcacccaaagttcatagcgtacagcagggttcactcttgggcagtacattccatgggtttggataaatgtgtaatgatgtctccaccatcacagcatcaggcagagtagtttcactgctctaacaaaatcctctgcctattcacccctctcattaaagccaaacactctgtttccttttttccttttagagacagtgtctcgctctgtcaaccaggctgaagtgcaatggcaatcacagcccattgcagcctccaactcctgggctcaagtgatcctcctatctcagcctccagtggctacgactgcaggcatacggcaacggcacccaactaattttttgtagagatagggtcttgctatgttgcccaggctggtcttgaactcttggtcctgccttagcctcccagagctctgggattacaggcgtgaaccaccgtgcccgtcccaaacactctgtttcgacctgcttttaaacaactgacccttggatgcattcaaaggatcagggtgtctgaaactggcctctgcagcaggaccttccttcctacacatctcccagtggccagtgtgaggattctccccacaagaaaccactggagggggcctcctcctgtccgggtttggggctgtacaaggagcatcatggacctggctcaggcctcaggaggggccctgggctggggaaaatgtgggatagcatcgaggcagtcccactcctacccagggccgggctagacctggggacagtctcagccatctcctcgctgcgtccacacaattccacccccacccccacccccaggctggccctcacggaagaacaacagctgatgtttgagaaactgactctgtattgcgacagctacatccagctcatccccatttccttcgtgctgggtgagttcccccttctggctgttccgggtccctgtggccgcccaggctccagacaggccaggggaggatcacgaggagctgcggcaaggggctggggagggggcgggggaacgccagcggcaggtcggcgcctctctgtagggaaaggtgcggactgcagccagagaaactgaagttagacgttaggtaagacgtcctgccgttagcaatgaaaaccccattttctgagggaagcgctgacatcatggtccctggagcccctgcgcgggaggggagggggtctggcggatttctgggaccagcagggggacccccgggtgacagaacccttggggctctcgcgcctccatgcgaggctctgcctgcctctcgctcccgagcgccttccaggagggctgggggctaggcccgctcgcagcagaaagctggaggagccgaggcatcgccgggcgctgggccctgggctctggccgcagcctggcccctcgcccctcgccccccgcccctcctgcccaggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggtgggcggaccgggagcaacggggaggcaccgggcagagccaggggccgagatgggcgcggcaggaacggaagatgggtggagccaaagtcccccggactcgggggactgggtggagccaggagtggggtgtggtcaagatttgggggtccaattgggcgggacagagtcgggtgtctgaaggtggggcgaggccaggagcccaccctccgagagtaggagtctgaggcagggctaaggacccttgagggataatggaaagaagggtgacggcttgggaactggtgaggtactagggtctacttccctctgcccttgcccctcttgatctccggtttccactctggaggtatgggacattggtctctgacaccccctcagcctggcctgacctggtcctggttaataagacagacccaggctaggcgtggtggctctcgcctgtaatcccagtgctttaggaggcaaaggtgggaagatcgcttgagcccagctgtttgagacgcccctgagcaacatagcgagacccccatctctacaaaaacattaaaaattagcagggcatggtggcgtgtgcctgtagtctgaggctgagtatcgggaggctgaggcaggaggatcacttgagcccagcagttccaggctgcagtgcgctaagatcgcaccgctgcactccaacctcggtgacagagccagaccctttctctggaaataaataaataccctgcccacatgctcagcccagaacagcacctagtaggtgctcagaaatttttttgttgttgaaagaaagaggatggcaaaggagtgctgaggttcctataggtcagcaggtgccggccatcccttctgcaggttctcccacccaccgccttcttcactccactctgcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgtgagcccactgtacagacagggctgccgcagagtgggaagggttgtggtccacaggaaacaaggtttcctacaaagagaagccttgggcccctgagggtcttccgagagccggaggtggggttgcagaatcttttccaacagcaatccacagcccgaggtggtcccttatcagaggcccctccctcttctccaagtctgtgaggtcctggttcccttttgatagatgaggaagctgagacacaaagaggtttagtgagcttcccatggccacacagccaggaatggaccataggtaccaggccctggtacctggagaagaggtgggggcgagcccagggtgggggcaggtggtgttcagaaccccatccccctcttctgccccccaggagatgaacaccttgcgtactcagtgtggacacctgtatgcctacgactggattagtatcccactggtgtatacacaggtgaggactaggctggtgaggctgcccttttgggaaactgaggctagaaggaccaaggaagcagctggggtgggaagggctcacctagaggctaagtggctcccctgggagttgggtccacactttgaagttgggtctggactttgaagtgccaagttctaagagtccaggctcctgcctggcccagtccagtagaggcaatgtgattatccccatattaaagagaggttggccgggcacagtggctcatgcctgtaatcccagcactttgggaagctgaggcaggtggatcacctgaggtcaggagttcgagaccagcctggccaacatggtgaaaccccatctctactgaaaatacagaattagctgtgtggtggtgcacgcctgtaatcccagctacttgggaggctgaggcaggagaatcgcttgaacccgggaggtggaggttgcagtgagctgagatcatgccactgcactccagcctgggcgacacagcaagactctgtctcaaacaaacaaacaaacaaacaaacaaacaaacaaacaaaggggttaacagagcccctaagtcacataagtgtgcaagtcagaacaaggccttggtctcctgtctcagactcccagcccctggagcatcctgatttcagggttcccacctagccctttgctaccacatcctcctcctcctcctcctcctcccaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtgggcctctccagggccctgctgggctggaggcatggccagaggggtcatggccagcagctgcttgagacgaggatgcagtgtcaggaaaggaaggtctcacgggtagaaagcagccaggcgtggtggcgcacacctgtaatcccagctactcgggaggctgaggcaggagaatcgcttgaacccgggaggcggaggttgtggtgagttgagatcgtgccactgcactccagcctgggcaaaagaatgaaactctatctcaaaaacaacaacaacaacaaaacaaagccctaaggttcagaagcccctgccctttagaagcagagcgaacactctcctattaagatgctgttgggtgtctttttcactcagtagctgtccagtattctccacacagcataatcgacagattctaatacaaatttcttcaactcttaattcctcctttgtgccaccattttttcttctacctcctaatttatgaatgggttagtatgctctgcttctgcattgagacaaaatacagagagagagaaagatctatcttaatcccgccccattttagttggaaaaaaactttattaaatcaggcaagtaaaatccgccaaggattgnnnnnnnnnnagatgttctgaatcagagagttttctctcgagctctttatctttccttccttctgttgcccacccactctctctcccttcctaccttcctttattttttggtaatgggggtgtaagtctctgtctctgcccttcctgtcactgtgacacacacacacacacacacacacacacacacacacacacacattcctattcctctaaattccccctgcacccccagttatctttggtttctgcagatcaaaacaaatcacacttttatgcttgaaattctccagggtgccccagtggcctgcaagatgtcccctggacccctaaggcagacgcgtgtcacctcttcggggctttgttagggcattttagaggttgctatccaggaatctgcccacctagactgccctttagttcagcccagcttcagtatatatctctgttgcatgaatgaataaaattatgcaactccaggtaagatacatgaggtgagataaaggcagtgactcagccgagtgatacactcagggacagctgtgggtgttcagggaaggactggctcagaagagttagaggggctgtgtccagaagtgtgtgggtgcctacaagtgtggggggctggagccctaaactctgcctttgaagacagtggtcaggcaggaagggcttcatggggtgtggaaatagcagcagctgaggtttaaagggggaagctggctttgaggagttctgcctgagggtttacagagcctcacctgtccccaaggtggcagagcagctcatcaacccctttggagaggatgatgatgattttgagaccaactggattgtcgacaggaatttgcaggtatggggagagggagagaaaccataccatggaccttccccaaagtggacccaaagagagctcctccctcctgcagccagtcattcactcacaggattctcacctcaatctttgaggctgcaggcaggcacccatctccccatttcacaggcagggaaactgaggtccagagagagggagagattcctccaagtcatcaggcacatacaaggtcctgcctgggatgatctttctgtgggacttcttctgtccctggtgaccaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcaggtgtggccagagccagggggctgggtgggaagcccctcctagtgcaggggtctgcctaggaacttagaatagcactagttaatgcatacaggttgcttcagtaagtgtcaggcactgtactatgctctttataaacattaactatttttttcctcccaataattctggtttgttatcccaagttttcagataattaaagtacaggttcagagagagtaagttgtccaaggccacatagctaccaaatggtgcatttgctactcgaaggacagcctatgatcagtgatgcagtggaacgttaggacctggctcttgtcatccagaactatgttttcttttctttttgagacagtatctcgctctgtcgcccaggttggagcgcagtggcgtgatcttggctcactgcaacctccgcctcctgggttcaagtgattctcctgcttcagcctccccagtagctgggattacaggtgcccacaaccacaactggctaatttttgtacttttagtagagatgaggtttcaccatgttggccaggctggtctccaactcctgaccagtaatctgcccgctttggcctcccaaaatgctggaattataggtgtcaaaactatgttttctgataagctacgatgcttggatgggaagtggaagtggggttccctgggatgggggaggggcagcaaagtcccagcaggcagccaggccatcacaggtacctcctgaattgactttgtcctaccgagtaaagggctcaggccacccacagcagccagacttatccccacatggtcccacttccctgattccatctgaatccctcttgagctgcagtgggctgaagggctatcccagctggtcctttctccccaggacaacagagttgaaagtgccttggagagtgttgggcacatgtcagggttcatactcaagggtttcttccacggtatccagtgctgttctcgcttgttcttttctttttttttttttaaacggagtttcactcttgttgcccagagctggagtgcagtggcataatctcggctcactgcaacctccgcctcccagattcaagcaattctcctgcctcagcctcctgagtagctgggattataggtgccagccaccaagcccggctaatttttgtatttttagtagagacagtttcaccatgttggccaggctggtctcgaactcctgacctcaggtgatccaccctcctcagcctcccaaagtgctgggattacatgtgtgagccactgtgcctggctgcttgttcttttaagaaccaaatatcctactagactgcaatcgagtttaactacagtctatagatactgtgaggaatggttgggaaggtcatcaaatgaaggctggaggcttgcttaggtcagaaacatttctggaggatgactttgagccctacatggtctgtaccccagcagctgaaggttgttgagggatggggagggctgaaaacagaacgataaagcatagaccttgtctccaaggaatgcacaatttatggagggagctcaaacccaagtctcaaactctggatacaaggtacaaagtactggatgtccagaaaagggacagaacatggaacacagtcatctttgtctgcctgggaggcggcttccagctgggtctggagctgagccatggaacatgggaagaatctgaacttgggcaagggcaggccatactctctggtagataagctttccttgcagggtaaaggtctggggctcccgggatgcctgttgctaggaagtcaaatttctctttgtggatgtcactcccagttggaaccacaaattcctggcattgcccagagtcactcatgggcctcatctgaaccactcatgccagggcaccagtgtttctgactgcctggagtgaggggttttacaggggaagtgaatgatgaggaggcctttacacgccaggcggggtggttgcgggggttggatgttaactctggtcaagagggaatcaacaaacagtgaggtgagctgggcctggagggatcaccgggaggtacagtacagatcaggagagaggtgagagctggggcatggtgaggaagacggtgtggccttggcttgggccaactgagagagaggagcgggggtaagggagaagtaaggccaggtgttggtcctttgtccactggctcagccctgcatctcctgtttctttccagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctgccccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacaggtctgtcctccacctgaaccaggggcactgcattgccctgtgccccaccccagcttcccttgctctgagcctacccttcctccacaatttcctagggttccatcactgccagagcacactggacctacgcccagcactggcttggggtatatacttggccaccttcacagggatcctagggaagtgttcgggaccttttctcacttcaccctggtatcacccggaagacttcttgggaccaggtgaaggaagatgaggttgtgctgaccagaatgctgctggagaactgccccagggctgacaggccaggcttagctgagcagatgttatcactggccccaacttactttgagcaagggtggctgacccaaaaccatgaggtggcagtcagctggatgacagatgaacacttcccccataactatttagggtagtacccaagcactacaggaaagggtggcaggaactgcctcactcctaggaactggtagatggtgaggttgagggtgtccagcgcccttaggtcattttctcactgcctgggaacctcaccaaaatacttcttgcttccttggggtcagcccaaagctgtcacaaaatcagatatttccctttattccagatttcctggacactgtcacccaattataaacaccccacttcagccccaatcacgtgggaggaagtgtaacttcccttttctggattctcaagcagttactttcacgggtcagaacacgcagctattatgattgaaaccttaaaagggcaacaatttcantcttgcttctaggctaagacaggaacttggcaaacatctgtggcctgttcagcaaaggatgttcatatttaagaatcttgtcttgggctgggtgtggaggcaagtgaatcacaggaggtcaggagtttgagaccaacctggccaacatgatgaaaccccatctctaccaaaaaaaatacaaatcagctggccgtcgtggtgtgcctgtagtcccaacgcaggaggttgaggggagaattgcttgaacccaggaggtggtggttgcagtgagattgagcaactgcaatccagcctgggcgacggagtgagactgtctcaaaaaaaaaaaaaaaaggatcgtctcaacctttgccctcctactgcaacattttggtatttgaaatgaaggtaccttccatacttatgctgttaatactttcattctcactagggatgaagcacattcctaacctgcttcctaatggggatgcttcgccagccaggtcctcacctgtgtgtacaccagcaggacactgatccagtcacagccatacagctgtccacactgaagaacgtgtcctacaacagcctgaatcaaatggttagcttaatagataaaaatcccagactacttcagcctttaatgccttttattcataaaaactgtgaaagctagactgaaccattggaaacatttaactcagactctggattcagagtcgggaacccttagttctatctgaatccaagacagccacaccttagtatactgcccaaactaatgagtttaataaatacaaatactcgt

SEQ ID NO: 2 - DNA, 2229 bp, Homo Sapiens
cagggagtcccaccagcctagtcgccagaccttctgtgggatcatcggacccacctggaaccccacctgacccaagcccacctgctgcagcccactgcctggccatgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagctacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctggcccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaacctgcttcctaatggggatgcttcgccagccaggtcctcacctgtgtgtacaccagcaggacactgatccagtcacagccatacagctgtccacactgaagaacgtgtcctacaacagcctgaatcaaatggttagcttaatagataaaaatcccagactacttcagcctttaatgccttttattcataaaaactgtgaaagctagactgaaccattggaaacatttaactcagactctggattcagagtcgggaacccttagttctatctgaatccaagacagccacaccttagtatactgcccaaactaatgagtttaataaatacaaatactcgttaaaaaaaaaaaaaaaaaaaaaaaaaaaa

SEQ ID NO: 3 - protein, 585 aa, Homo Sapiens
MTITYTSQVANARLGSFSRLLLCWRGSIYKLLYGEFLIFLLCYYIIRFIYRLALTEEQQLMFEKLTLYCDSYIQLIPISFVLGFYVTLVVTRWWNQYENLPWPDRLMSLVSGFVEGKDEQSRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVAEQLINPFGEDDDDFETNWIVDRNLQVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSGPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRDEAHS

SEQ ID NO: 4 - DNA, 2429 bp, Homo Sapiens
cagggagtcccaccagcctagtcgccagaccttctgtgggatcatcggacccacctggaaccccacctgacccaagcccacctgctgcagcccactgcctggccatgaccatcacttacacaagccaagtggctaatgcccgcttaggctccttctcccgcctgctgctgtgctggcggggcagcatctacaagctgctatatggcgagttcttaatcttcctgctctgctactacatcatccgctttatttataggctggccctcacggaagaacaacagctgatgtttgagaaactgactctgtattgcgacagctacatccagctcatccccatttccttcgtgctgggcttctacgtgacgctggtcgtgacccgctggtggaaccagtacgagaacctgccgtggcccgaccgcctcatgagcctggtgtcgggcttcgtcgaaggcaaggacgagcaaggccggctgctgcggcgcacgctcatccgctacgccaacctgggcaacgtgctcatcctgcgcagcgtcagcaccgcagtctacaagcgcttccccagcgcccagcacctggtgcaagcaggctttatgactccggcagaacacaagcagttggagaaactgagcctaccacacaacatgttctgggtgccctgggtgtggtttgccaacctgtcaatgaaggcgtggcttggaggtcgaatccgggaccctatcctgctccagagcctgctgaacgagatgaacaccttgcgtactcagtgtggacacctgtatgcctacgactggattagtatcccactggtgtatacacaggtggtgactgtggcggtgtacagcttcttcctgacttgtctagttgggcggcagtttctgaacccagccaaggcctaccctggccatgagctggacctcgttgtgcccgtcttcacgttcctgcagttcttcttctatgttggctggctgaaggtgggcctctccagggccctgctgggctggaggcatggccagaggggtcatggccagcagctgcttgagacgaggatgcagtgtcaggaaaggaaggtctcacgggtagaaagcagccaggcgtggtggcgcacacctgtaatcccagctactcgggaggctgaggcaggagaatcgcttgaacccgggaggcggaggttgtggtggcagagcagctcatcaacccctttggagaggatgatgatgattttgagaccaactggattgtcgacaggaatttgcaggtgtccctgttggctgtggatgagatgcaccaggacctgcctcggatggagccggacatgtactggaataagcccgagccacagcccccctacacagctgcttccgcccagttccgtcgagcctcctttatgggctccaccttcaacatcagcctgaacaaagaggagatggagttccagcccaatcaggaggacgaggaggatgctcacgctggcatcattggccgcttcctaggcctgcagtcccatgatcaccatcctcccagggcaaactcaaggaccaaactactgtggcccaagagggaatcccttctccacgagggcctgcccaaaaaccacaaggcagccaaacagaacgttaggggccaggaagacaacaaggcctggaagcttaaggctgtggacgccttcaagtctggcccactgtatcagaggccaggctactacagtgccccacagacgcccctcagccccactcccatgttcttccccctagaaccatcagcgccgtcaaagcttcacagtgtcacaggcatagacaccaaagacaaaagcttaaagactgtgagttctggggccaagaaaagttttgaattgctctcagagagcgatggggccttgatggagcacccagaagtatctcaagtgaggaggaaaactgtggagtttaacctgacggatatgccagagatccccgaaaatcacctcaaagaacctttggaacaatcaccaaccaacatacacactacactcaaagatcacatggatccttattgggccttggaaaacagggatgaagcacattcctaacctgcttcctaatggggatgcttcgccagccaggtcctcacctgtgtgtacaccagcaggacactgatccagtcacagccatacagctgtccacactgaagaacgtgtcctacaacagcctgaatcaaatggttagcttaatagataaaaatcccagactacttcagcctttaatgccttttattcataaaaactgtgaaagctagactgaaccattggaaacatttaactcagactctggattcagagtcgggaacccttagttctatctgaatccaagacagccacaccttagtatactgcccaaactaatgagtttaataaatacaaatactcgttaaaaaaaaaaaaaaaaaaaaaaaaa

SEQ ID NO: 5 - protein, 435 aa, Homo Sapiens [sequence omitted]

SEQ ID NO: 6 - DNA, 18 bp, Homo Sapiens
cagggagtcccaccagcc

SEQ ID NO: 7 - DNA, 18 bp, Homo Sapiens
tccccattaggaagcagg

SEQ ID NO: 8 - DNA, 18 bp, Homo Sapiens
tctcctctttgttcaggc

SEQ ID NO: 9 - DNA, 21 bp, Homo Sapiens
ctagtcgccagaccttctgtg

SEQ ID NO: 10 - DNA, 20 bp, Homo Sapiens
cttgtagactgcggtgctga

SEQ ID NO: 11 - DNA, 20 bp, Homo Sapiens
gaaagcaaggacgagcaaag

SEQ ID NO: 12 - DNA, 22 bp, Homo Sapiens
aatccagtcgtaggcatacagg

SEQ ID NO: 13 - DNA, 21 bp, Homo Sapiens
accttgcgtactcagtgtgga

SEQ ID NO: 14 - DNA, 21 bp, Homo Sapiens
tgtcgacaatccagttggtct

SEQ ID NO: 15 - DNA, 20 bp, Homo Sapiens
ccctttggagaggatgatga

SEQ ID NO: 16 - DNA, 20 bp, Homo Sapiens
ctctggcatatccgtcaggt

SEQ ID NO: 17 - DNA, 20 bp, Homo Sapiens
cttcaagtctgccccactgt

SEQ ID NO: 18 - DNA, 20 bp, Homo Sapiens
gcatccccattaggaagcag

SEQ ID NO: 19 - DNA, 20 bp, Homo Sapiens
ctaagcgggcattagccact

SEQ ID NO: 20 - DNA, 22 bp, Homo Sapiens
tggggttccaggtgggtccgat

SEQ ID NO: 21 - DNA, 27 bp, Homo Sapiens
ccatcctaatacgactcactatagggc

SEQ ID NO: 22 - DNA, 27 bp, Homo Sapiens
ggatgaagcacattcctaacctgcttc

SEQ ID NO: 23 - DNA, 18 bp, Homo Sapiens
aaagctggaggagccgag

SEQ ID NO: 24 - DNA, 19 bp, Homo Sapiens
ctccacccatcttccgttc

SEQ ID NO: 25 - DNA, 20 bp, Homo Sapiens
taggctcagagcaagggaag

SEQ ID NO: 26 - DNA, 20 bp, Mus Musculus
acacaacacattctgggtgc

SEQ ID NO: 27 - DNA, 20 bp, Mus Musculus
ttcagaaactgcttcccgat

SEQ ID NO: 28 - DNA, 1916 bp, Mus Musculus
gtgccaagccatgactatcacctacacaaacaaagtagccaatgcccgcctcggttcgttctcgtccctcctcctgtgctggcgaggcagcatctacaagctgctgtatggagaattccttgtcttcatattcctctactattccatccgtggactctacagaatggttctctcgagtgatcagcagctgttgtttgagaagctggctctgtactgcgacagctacattcagctcatccctatatccttcgttctgggtttctatgttacattggtggtgagccgctggtggagccagtacgagaacttgccgtggcccgaccgcctcatgatccaggtgtctagcttcgtggagggcaaggatgaggaaggccgtttgctgcggcgcacgctcatccgctacgccatcctgggccaagtgctcatcctgcgcagcatcagcacctcggtctacaagcgctttcccactcttcaccacctggtgctagcaggttttatgacccatggggaacataagcagttgcagaagttgggcctaccacacaacacattctgggtgccctgggtgtggtttgccaacttgtcaatgaaggcctatcttggaggtcgaatccgggacaccgtcctgctccagagcctgatgaatgaggtgtgtactttgcgtactcagtgtggacagctgtatgcctacgactggataagtatcccattggtgtacacacaggtggtgacagtggcagtatacagctttttccttgcatgcttgatcgggaggcagtttctgaacccaaacaaggactacccaggccatgagatggatctggttgtgcctgtcttcacaatcctgcaattcttattctacatgggctggctgaaggtggcagaacagctcatcaaccccttcggggaggacgatgatgattttgagactaactggatcattgacagaaacctgcaggtgtccctgttgtccgtggatgggatgcaccagaacttgcctcccatggaacgtgacatgtactggaacgaggcagcgcctcagccgccctacacagctgcttctgccaggtctcgccggcattccttcatgggctccaccttcaacatcagcctaaagaaagaagacttagagctttggtcaaaagaggaggctgacacggataagaaagagagtggctatagcagcaccataggctgcttcttaggactgcaacccaaaaactaccatcttcccttgaaagacttaaagaccaaactattgtgttctaagaaccccctcctcgaaggccagtgtaaggatgccaaccagaaaaaccagaaagatgtctggaaatttaagggtctggacttcttgaaatgtgttccaaggtttaagaggagaggctcccattgtggcccacaggcacccagcagccaccctactgagcagtcagcaccctccagttcagacacaggtgatgggccttccacagattaccaagaaatctgtcacatgaaaaagaaaactgtggagtttaacttgaacattccagagagccccacagaacatcttcaacagcgccgtttggaccagatgtcaaccaatatacaggctctaatgaaggagcatgcagagtcctatccctacagggatgaagctggcaccaaacctgttctctatgagtgatgcctcacagcctggccctgacttgcaaggatgcccagcagggcactgacccagtcaaaggcacacaagcagcgacacccaggagtgtgttcccacgacagtctagcatgtaactcagaaccaagagtacttaatagtcctgcctgaaaacacctgtattttacgatctttcccaaactaaggagtttaataaacgtgaatattcttttaggtgaaaaaaaaaaaaaaaaaaaaaaaaaaaaaaaaaaaa

SEQ ID NO: 29 - protein, 551 aa, Mus Musculus
MTITYTNKVANARLGSFSSLLLCWRGSIYKLLYGEFLVFIFLYYSIRGLYRMVLSSDQQLLFEKLALYCDSYIQLIPISFVLGFYVTLVVSRWWSQYENLPWPDRLMIQVSSFVEGKDEEGRLLRRTLIRYAILGQVLILRSISTSVYKRFPTLHHLVLAGFMTHGEHKQLQKLGLPHNTFWVPWVWFANLSMKAYLGGRIRDTVLLQSLMNEVCTLRTQCGQLYAYDWISIPLVYTQVVTVAVYSFFLACLIGRQFLNPNKDYPGHEMDLVVPVFTILQFLFYMGWLKVAEQLINPFGEDDDDFETNWIIDRNLQVSLLSVDGMHQNLPPMERDMYWNEAAPQPPYTAASARSRRHSFMGSTFNISLKKEDLELWSKEEADTDKKESGYSSTIGCFLGLQPKNYHLPLKDLKTKLLCSKNPLLEGQCKDANQKNQKDVWKFKGLDFLKCVPRFKRRGSHCGPQAPSSHPTEQSAPSSSDTGDGPSTDYQEICHMKKKTVEFNLNIPESPTEHLQQRRLDQMSTNIQALMKEHAESYPYRDEAGTKPVLYE

SEQ ID NO: 30 - DNA, 19 bp, Homo Sapiens
ctcctgcccaggcttctac

SEQ ID NO: 31 - DNA, 19 bp, Homo Sapiens
cttgctctgccttgccttc

SEQ ID NO: 32 - protein, 30 aa, Homo Sapiens
IPISFVLGFYVTLVVTRWWNQYENLPWPDR

SEQ ID NO: 33 - protein, 30 aa, C. elegans
IPLTFMLGFFVTIIVGRWNDIFLNIGWVDN

SEQ ID NO: 34 - protein, 30 aa, C. elegans
IPLTFMLGFFVTIIVRRWNDIFANLGWVEN

SEQ ID NO: 35 - protein, 30 aa, C. elegans
IPLEFVLGFFVTIVVDRWTKLWRTVGFIDD

SEQ ID NO: 36 - protein, 30 aa, C. elegans
IPLEFVLGFFVTTVVNRWTKLYQTIGFIDN

SEQ ID NO: 37 - protein, 30 aa, C. elegans
VPLDWMLGFFIAGVLRRFWYLYDIIGFIDN

SEQ ID NO: 38 - protein, 30 aa, C. elegans
IPLNFMLGFFVTAVVNRWTYLYQIIGFIDN

SEQ ID NO: 39 - protein, 30 aa, C. elegans
LPLNFVLGFFCNIIIRRWLKLYTSLGNIDN

SEQ ID NO: 40 - protein, 30 aa, C. elegans
IPINFMLGFFVTTVINRWMTQFANLGMIDN

SEQ ID NO: 41 - protein, 30 aa, C. elegans
IPLTFLLGFFVSFVVARWGSILNGIGWIDD

SEQ ID NO: 42 - protein, 30 aa, C. elegans
IPVTFMLGFYVSIVYNRWTKVFDNVGWIDT

SEQ ID NO: 43 - protein, 30 aa, C. elegans
LPLTFMLGFFVTTVFERWRSALNVMPFIES

SEQ ID NO: 44 - protein, 30 aa, C. elegans
IPLTFLLGFYVSNVVSRWWRQFETLRWPED

SEQ ID NO: 45 - protein, 30 aa, C. elegans
IPLTFLLGFYVSNVVARWWRQFETLYWPED

SEQ ID NO: 46 - protein, 30 aa, C. elegans
IPLTFLLGFYVAMIVRRWWDCCQLISWPDH

SEQ ID NO: 47 - protein, 30 aa, C. elegans
IPLSFLLGFFVSLIVARWWEQFNCISWPDK

SEQ ID NO: 48 - protein, 30 aa, C. elegans
VPMQPMLGYFIGMVGERWGESFENVSYIEK

(19) United States
(12) Patent Application Publication
(10) Pub. No.: US 2006/0105364 A1
(43) Pub. Date: May 18, 2006

(54) Best's macular dystrophy gene

(76) Inventors: Konstantin Petrukhin, Collegeville, PA (US); C. Thomas Caskey, Lansdale, PA (US); Michael Metzker, Fort Washington, PA (US); Claes Wadelius, Upsala (SE)

Correspondence Address: MERCK AND CO., INC, P O BOX 2000, RAHWAY, NJ, 07065-0907, US

(21) Appl. No.: 11/236,238

(22) Filed: Sep. 27, 2005

Related U.S. Application Data
(62) Division of application No. 09/622,964, filed on Dec. 12, 2000, now Pat. No. 7,005,290.
(60) Provisional application No. 60/075,941, filed on Feb. 25, 1998.
(60) Provisional application No. 60/112,926, filed on Dec. 18, 1998.

(51) Int. Cl. C12Q 1/68; C07H 21/04; C12P 21/06; C07K 14/705

(52) U.S. Cl. 435/6; 435/69.1; 435/320.1; 435/325; 530/350; 536/23.2

(57) ABSTRACT

Novel human and mouse DNA sequences that encode the gene CG1CE, which, when mutated, is responsible for Best's macular dystrophy, are provided. Provided are genomic CG1CE DNA as well as cDNA that encodes the CG1CE protein. Also provided is CG1CE protein encoded by the novel DNA sequences. Methods of expressing CG1CE protein in recombinant systems are provided. Also provided are diagnostic methods that detect patients having mutant CG1CE genes.

### CROSS-REFERENCE TO RELATED APPLICATIONS

This application claims the benefit of U.S. application Ser. No. 09/622,964 filed Dec. 12, 2000 and PCT Application No. PCT/US99/03790 filed 22 Feb. 1999 which is based upon U.S. Provisional Application No. 60/075,941 filed Feb. 25, 1998 and U.S. Provisional Application No. 60/122,926 filed Dec. 18, 1998, the contents of which are incorporated herein by reference in their entirety.

### STATEMENT REGARDING FEDERALLY-SPONSORED R&D

Not applicable.

### REFERENCE TO MICROFICHE APPENDIX

Not applicable.

### FIELD OF THE INVENTION

The present invention is directed to novel human and mouse DNA sequences encoding a protein which, when present in mutated form, results in the occurrence of Best's Macular Dystrophy.

### BACKGROUND OF THE INVENTION

Macular dystrophy is a term applied to a heterogeneous group of diseases that collectively are the cause of severe visual loss in a large number of people. A common characteristic of macular dystrophy is a progressive loss of central vision resulting from the degeneration of the pigmented epithelium underlying the retinal macula. In many forms of macular dystrophy, the end stage of the disease results in legal blindness. More than 20 types of macular dystrophy are known: e.g., age-related macular dystrophy, Stargardt's disease, atypical vitelliform macular dystrophy (VMD1), Usher Syndrome Type 1B, autosomal dominant neovascular inflammatory vitreoretinopathy, familial exudative vitreoretinopathy, and Best's macular dystrophy (also known as hereditary macular dystrophy or Best's vitelliform macular dystrophy (VMD2)). For a review of the macular dystrophies, see Sullivan & Daiger, 1996, Mol. Med. Today 2:380-386.

Best's Macular Dystrophy (BMD) is an inherited autosomal dominant macular dystrophy of unknown biochemical cause. BMD has an age of onset that can range from childhood to after 40. Clinical symptoms include, at early stages, an abnormal accumulation of the yellowish material lipofuscin in the retinal pigmented epithelium (RPE) underlying the macula. This gives rise to a characteristic “egg yolk” appearance of the RPE and gradual loss of visual acuity. With increasing age, the RPE becomes more and more disorganized, as the lipofuscin accumulations disperse and scarring and neovascularization take place. These changes are accompanied by further loss of vision.

The pathological features seen in BMD are in many ways similar to the features seen in age-related macular dystrophy, the leading cause of blindness in older patients in the developed world. Age-related macular dystrophy is an extraordinarily difficult disease to study genetically, since by the time patients are diagnosed, their parents are usually no longer living and their children are still asymptomatic. Thus, family studies which have led to the discovery of the genetic basis of many other diseases have not been practical for age-related macular dystrophy. As there are currently no widely effective treatments for age-related macular dystrophy, it is hoped that study of BMD, and in particular the discovery of the underlying genetic cause of BMD, will shed light on age-related macular dystrophy as well.

Linkage analysis has established that the gene responsible for BMD resides in the pericentric region of chromosome 11, at 11q13, near the markers D11S956, FCER1B, and UGB (Forsman et al., 1992, Clin. Genet. 42:156-159; Hou et al., 1996, Human Heredity 46:211-220). Recently, the gene responsible for BMD was localized to a ˜1.7 mB PAC contig lying mostly between the markers D11S1765 and UGB (Cooper et al., 1997, Genomics 41:185-192). Recombination breakpoint mapping in a large Swedish pedigree limited the minimum genetic region containing the BMD gene to a 980 kb interval flanked by the microsatellite markers D11S4076 and UGB (Graff et al., 1997, Hum. Genet. 101: 263-279).

One difficulty in diagnosing BMD is that carriers of the diseased gene for BMD may be asymptomatic in terms of visual acuity and morphological changes of the RPE observable in a routine ophthalmologic examination. There does exist a test, the electro-oculographic examination (EOG), which detects differences in electrical potential between the cornea and the retina, that can distinguish asymptomatic BMD patients from normal individuals. However, the EOG requires specialized, expensive equipment, is difficult to administer, and requires that the patient be present at the site of the equipment when the test is performed. It would be valuable to have an alternative method of diagnosing asymptomatic carriers of mutations in the gene responsible for BMD that is simpler, less expensive, and does not require the presence of the patient while the test is being performed. For example, a diagnostic test that relies on a blood sample from a patient suspected of being an asymptomatic carrier of BMD would be ideal.

### SUMMARY OF THE INVENTION

The present invention is directed to novel human and mouse DNA sequences that encode the gene CG1CE, which, when mutated, is responsible for Best's macular dystrophy. The present invention includes genomic CG1CE DNA as well as cDNA that encodes the CG1CE protein. The human genomic CG1CE DNA is substantially free from other nucleic acids and has the nucleotide sequence shown in SEQ.ID.NO.:1. The human cDNA encoding CG1CE protein is substantially free from other nucleic acids and has the nucleotide sequence shown in SEQ.ID.NO.:2 or SEQ.ID.NO.:4. The mouse cDNA encoding CG1CE protein is substantially free from other nucleic acids and has the nucleotide sequence shown in SEQ.ID.NO.:28. Also provided is CG1CE protein encoded by the novel DNA sequences. The human CG1CE protein is substantially free from other proteins and has the amino acid sequence shown in SEQ.ID.NO.:3 or SEQ.ID.NO.:5. The mouse CG1CE protein is substantially free from other proteins and has the amino acid sequence shown in SEQ.ID.NO.:29. Methods of expressing CG1CE protein in recombinant systems are provided. Also provided are diagnostic methods that detect carriers of mutant CG1CE genes.

### BRIEF DESCRIPTION OF THE DRAWINGS

FIG. 1A-F shows the genomic DNA sequence of human CG1CE (SEQ.ID.NO.:1). Underlined nucleotides in capitals represent exons. The start ATG codon in exon 2 and the stop TAA codon in exon 11 are shown in bold italics. The consensus polyadenylation signal AATAAA in exon 11 is shown in bold. The alternatively spliced part of exon 7 is shown in underlined italics. The exact lengths of two gaps between exons 1 and 2 and between exons 7 and 8 are unknown; these gaps are presented as runs of ten Ns for the sake of convenience. The portion of exon 111 beginning at position 15,788 represents the 3′ untranslated region; 132 base pairs downstream of the polyadenylation signal of the CG1CE gene are multiple ESTs, representing the 3′-untranslated region of the ferritin heavy chain gene (FTH). FTH has been mapped to human chromosome 11q13 (Hentze et al., 1986, Proc. Nat. Acad. Sci. 83: 7226-7230); the FTH gene was later shown to be a part of the smallest minimum genetic region containing the BMD gene, as determined by recombination breakpoint mapping in a 12 generation Swedish pedigree (Graff et al., 1997, Hum. Genet. 101: 263-279).

FIG. 2 shows the complete sequence of the short form of human CG1CE cDNA (SEQ.ID.NO.:2). The ATG start codon is at position 105; the TAA stop codon is at position 1,860.

FIG. 3 shows the complete amino acid sequence of the long form of human CG1CE protein (SEQ.ID.NO.:3). This long form of the human CG1CE protein is produced by translation of the short form of CG1CE cDNA.

FIG. 4 shows the complete sequence of the long form of human CG1CE cDNA (SEQ.ID.NO.:4). This long form of the human CG1CE cDNA is produced when an alternative splice donor site is utilized in intron 7. The ATG start codon is at position 105; the TGA stop codon is at position 1410.

FIG. 5 shows the complete amino acid sequence of the short form of the human CG1CE protein (SEQ.ID.NO.:5). This short form of the human CG1CE orotein is produced by translation of the long form of CG1CE cDNA.

FIG. 6 shows the results of sequencing runs of PCR fragments that represent exon 4 and adjacent intronic regions from three individuals from the Swedish pedigree S1, two of whom are affected with BMD. From top to bottom, the runs are: patient S1-5 (homozygous affected with BMD), sense orientation; patient S1-4 (heteroozygous affected with BMD), sense orientation; patient S1-3 (normal control, unaffected sister of S1-4), sense orientation; patient S 1-5 (affected with BMD), anti-sense orientation; patient S1-4 (affected with BMD), anti-sense orientation; patient S1-3 (normal control), anti-sense orientation. Reading from left to right, the mutation shows up at position 31 of the sequence shown in the case of patients S1-5 and S1-4. The mutation in family S1 changes tryptophan to cysteine.

FIG. 7 shows a multiple sequence alignment of human CG1CE protein with partial sequences of related proteins from C. elegans. Related proteins from C. elegans were identified by BLASTP analysis of non-redundant GenBank database. This figure shows that two amino acids mutated in two different Swedish families with BMD (families S1 and SL76) are evolutionarily conserved. 15 of 16 related proteins from C. elegans contain a tryptophan at the position of the mutation in family S1, as does the wild-type CG1CE gene. Only one C. elegans protein does not have a tryptophan at the position of the mutation. In this protein (accession number p34577), tryptophan is changed for isofunctional phenylalanine (phenylalanine is highly similar to tryptophan in that it also is a hydrophobic aromatic amino acid). Mutation in the BMD family SL76 changes a tyrosine to histidine. Again, all 16 related proteins from C. elegans contain tyrosine or isofunctional phenylalanine in this position (tyrosine is highly similar to phenylalanine in that it also is an aromatic amino acid).

FIG. 8A-C shows the complete sequence of mouse CG1CE cDNA (SEQ.ID.NO.:28) and mouse CG1CE protein (SEQ.ID.NO.:29).

FIG. 9A-B shows an alignment of the amino acid sequences of the long form of human CG1CE protein (SEQ.ID.NO.:3) and mouse CG1CE protein (SEQ.ID.NO.:29). In this figure, CG1CE is referred to as “bestrophin.”

FIG. 10A-C shows the results of in situ hybridization experiments demonstrating that mouse CG1CE mRNA expression is localized to the retinal pigmented epithelium cells (RPE). FIG. 10A shows the results of using an antisense CG1CE probe. The antisense probe hybridizes to mouse CG1CE mRNA present in the various cell layers of the retina, labeling with dark bands the cells containing CG1CE mRNA. The antisense probe strongly hybridized to the RPE cells and not to the cells of the other layers of the retina. FIG. 10B shows the results using a sense CG1CE probe as a control. The sense probe does not hybridize to CG1CE mRNA and does not label the RPE cells. FIG. 10C is a higher magnification of the RPE cells from FIG. 10A. Human CG1CE mRNA shows a similar distribution, being confined to the RPE cells of the human retina.

### DETAILED DESCRIPTION OF THE INVENTION

For the purposes of this invention:

“Substantially free from other proteins” means at least 90%, preferably 95%, more preferably 99%, and even more preferably 99.9%, free of other proteins. Thus, a CG1CE protein preparation that is substantially free from other proteins will contain, as a percent of its total protein, no more than 10%, preferably no more than 5%, more preferably no more than 1%, and even more preferably no more than 0.1%, of non-CG1CE proteins. Whether a given CG1CE protein preparation is substantially free from other proteins can be determined by such conventional techniques of assessing protein purity as, e.g., sodium dodecyl sulfate polyacrylamide gel electrophoresis (SDS-PAGE) combined with appropriate detection methods, e.g., silver staining or immunoblotting.

“Substantially free from other nucleic acids” means at least 90%, preferably 95%, more preferably 99%, and even more preferably 99.9%, free of other nucleic acids. Thus, a CG1CE DNA preparation that is substantially free from other nucleic acids will contain, as a percent of its total nucleic acid, no more than 10%, preferably no more than 5%, more preferably no more than 1%, and even more preferably no more than 0.1%, of non-CG1CE nucleic acids. Whether a given CG1CE DNA preparation is substantially free from other nucleic acids can be determined by such conventional techniques of assessing nucleic acid purity as, e.g., agarose gel electrophoresis combined with appropriate staining methods, e.g., ethidium bromide staining, or by sequencing.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid); substitution of one aromatic amino acid (tryptophan, tyrosine, or phenylalanine) for another.

The present invention relates to the identification and cloning of CG1CE, a gene which, when mutated, is responsible for Best's macular dystrophy. That CG1CE is the Best's macular dystrophy gene is supported by various observations:

1. CG1CE maps to the genetically defined region of human chromosome 11q 2-q 13 that has been shown to contain the Best's macular dystrophy gene. CG1CE is present on two PAC clones, 759J12 and 466A11, that lie precisely in the most narrowly defined region that has been shown to contain CG1CE (Cooper et al., 1997, Genomics 41:185-192; Stöhr et al., 1997, Genome Res. 8:48-56; Graff et al., 1997, Hum. Genet. 101: 263-279).

2. CG1CE is expressed predominately in the retina.

3. In patients having Best's macular dystrophy, CG1CE contains mutations in evolutionarily conserved amino acids.

4. The CG1CE genomic clones contain another gene (FTH) that has been physically associated with the Best's macular dystrophy region (Cooper et al., 1997, Genomics 41:185-192; Stöhr et al., 1997, Genome Res. 8:48-56; Graff et al., 1997, Hum. Genet. 101:263-279). The FTH and CG1CE genes are oriented tail-to-tail; the distance between their polyadenylation signals is 132 bp.

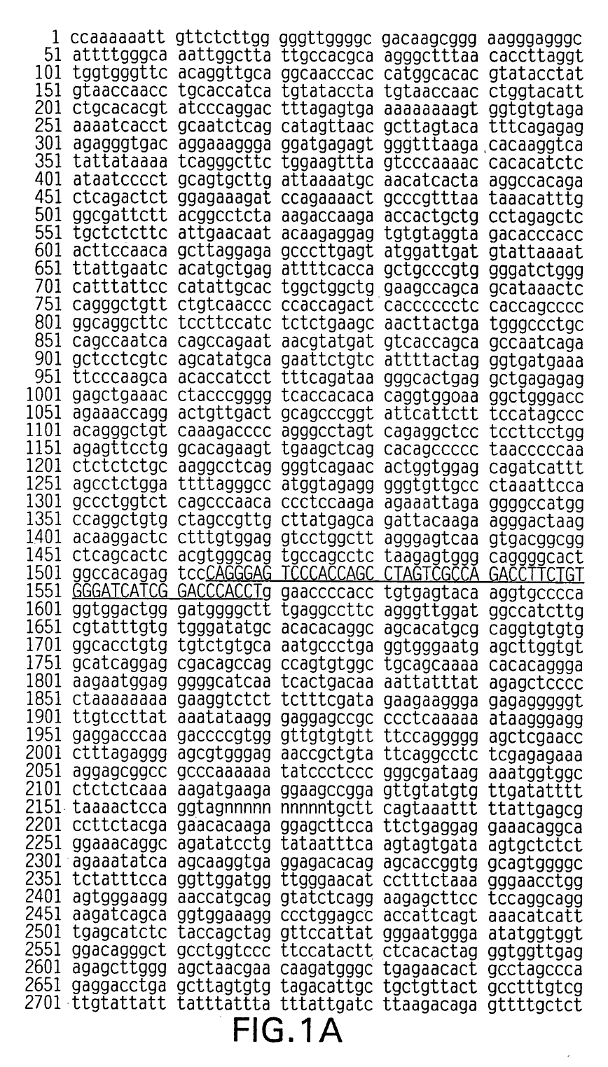

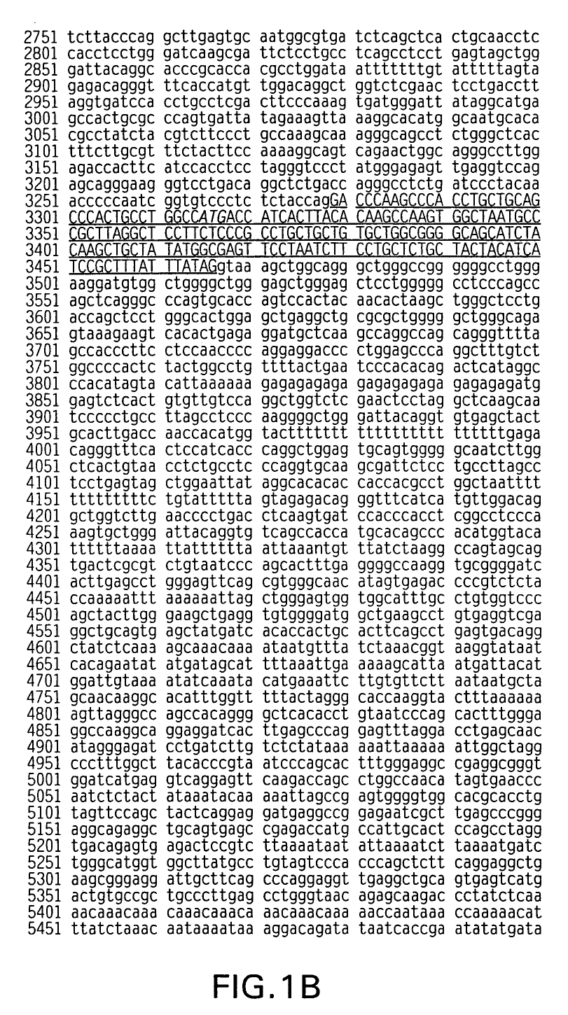

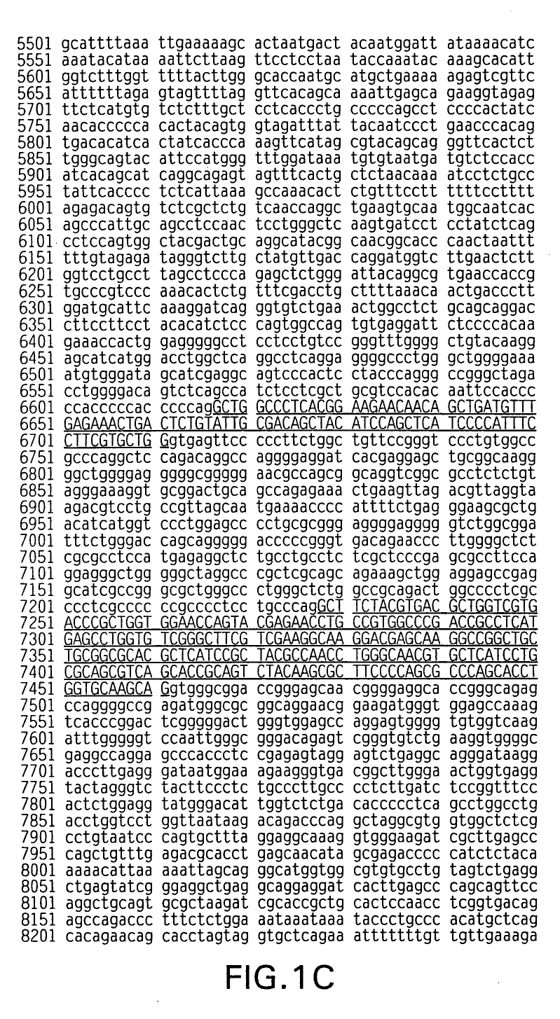

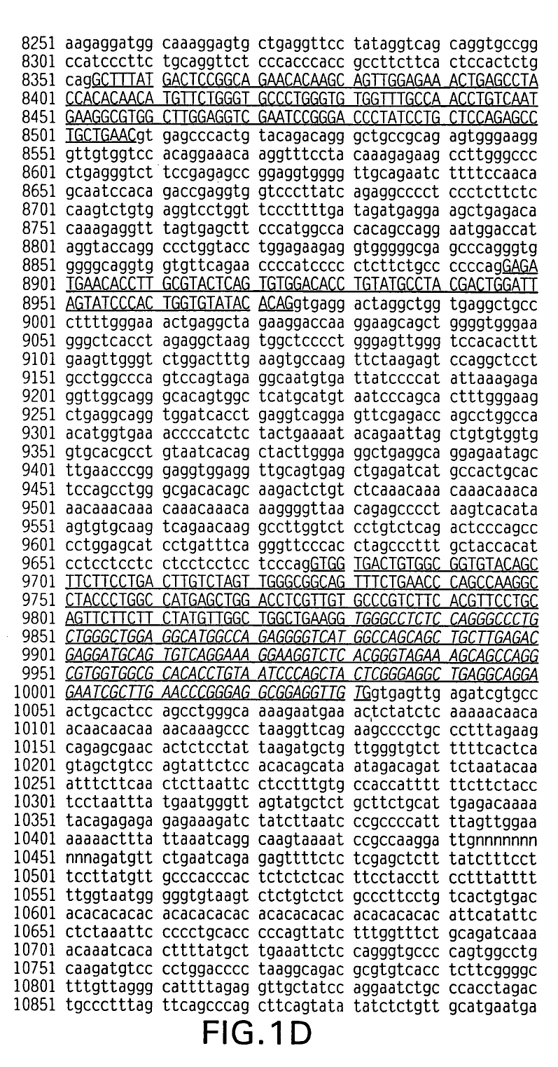

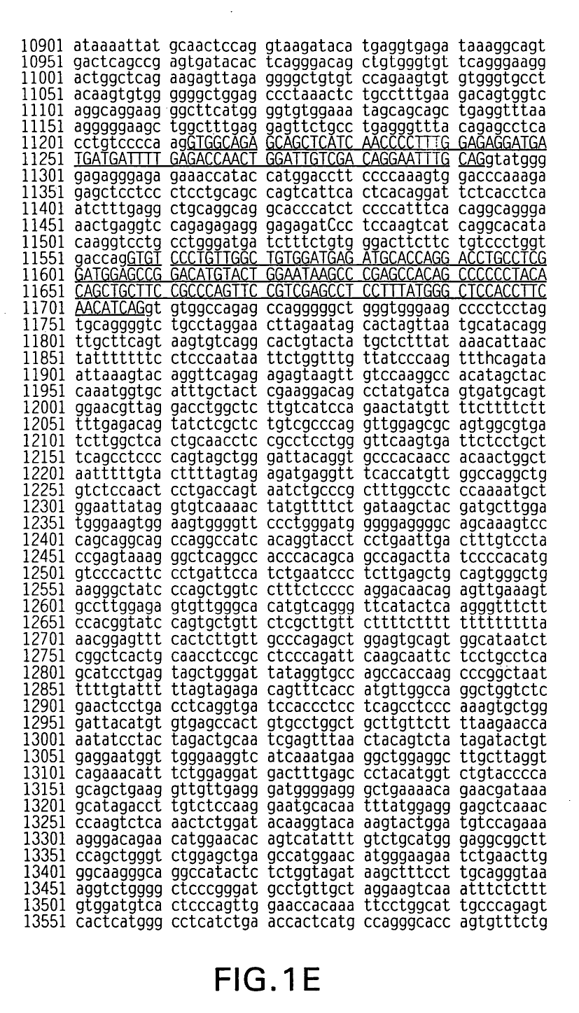

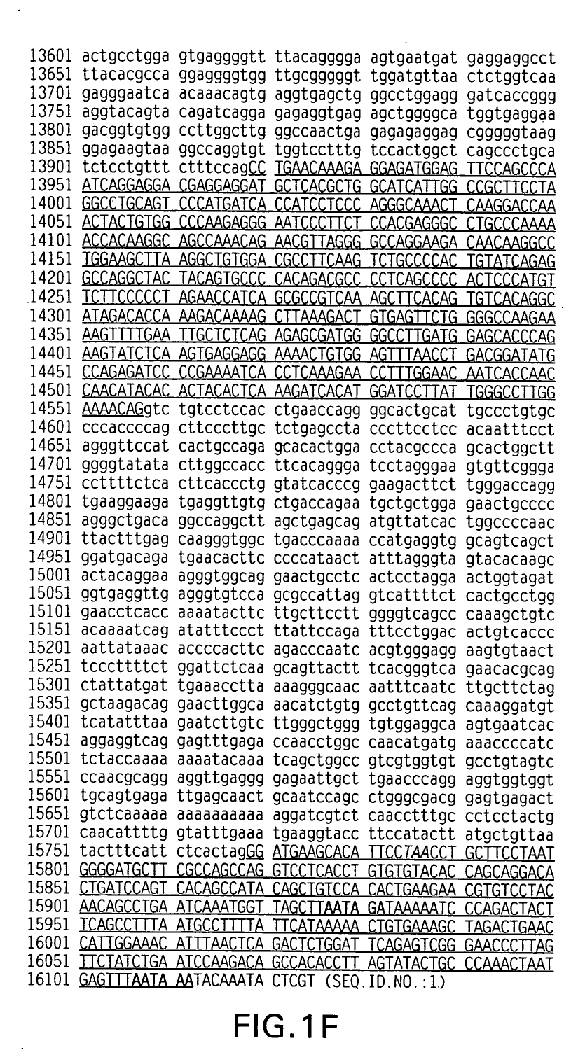

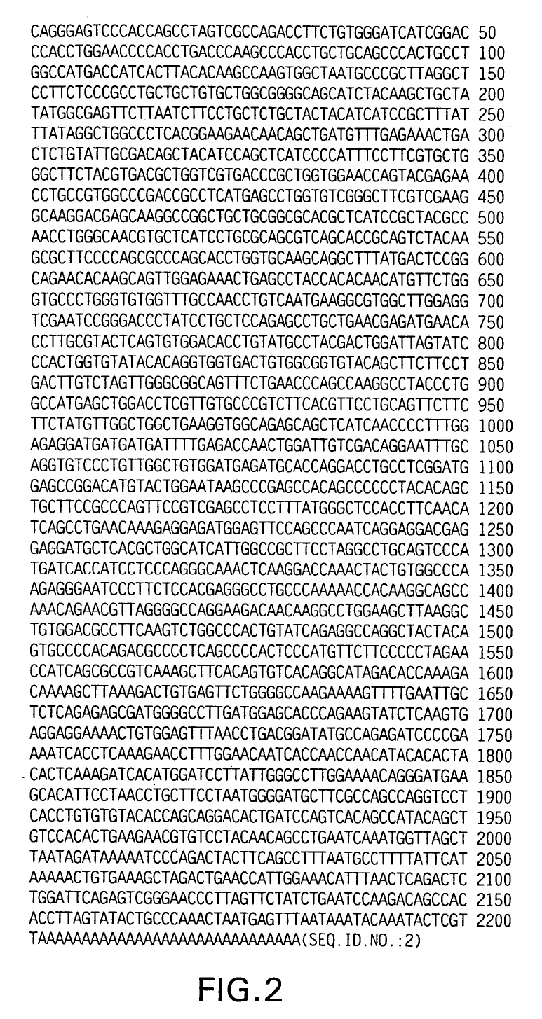

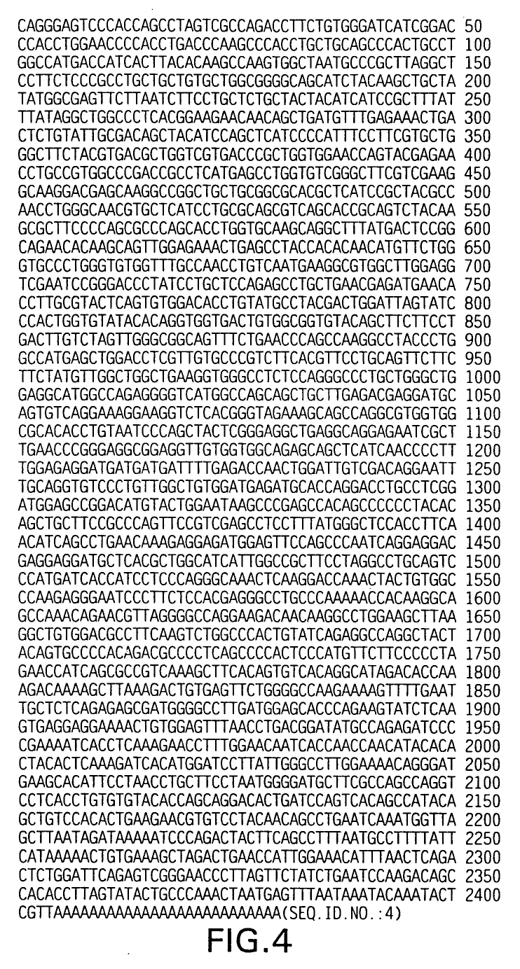

The present invention provides DNA encoding CG1CE that is substantially free from other nucleic acids. The present invention also provides recombinant DNA molecules encoding CG1CE. The present invention provides DNA molecules substantially free from other nucleic acids comprising the nucleotide sequence shown in FIG. 1 as SEQ.ID.NO.:1. Analysis of SEQ.ID.NO.:1 revealed that this genomic sequence defines a gene having 11 exons. These exons collectively have an open reading frame that encodes a protein of 585 amino acids. If an alternative splice donor site is utilized in exon 7, a cDNA containing an additional 203 bases is produced. Although longer, this cDNA contains a shorter open reading frame of 1,305 bases (due to the presence of a change in reading frame that introduces a stop codon) that encodes a protein of 435 amino acids. Thus, the present invention includes two cDNA molecules encoding two forms of CG1CE protein that are substantially free from other nucleic acids and have the nucleotide sequences shown in FIG. 2 as SEQ.ID.NO.:2 and in FIG. 4 as SEQ.ID.NO.:4.

The present invention includes DNA molecules substantially free from other nucleic acids comprising the coding regions of SEQ.ID.NO.:2 and SEQ.ID.NO.:4. Accordingly, the present invention includes DNA molecules substantially free from other nucleic acids having a sequence comprising positions 105-1,859 of SEQ.ID.NO.:2 and positions 105-1,409 of SEQ.ID.NO.:4. Also included are recombinant DNA molecules having a nucleotide sequence comprising positions 105-1,859 of SEQ.ID.NO.:2 and positions 105-1,409 of SEQ.ID.NO.:4.

Portions of the cDNA sequences of SEQ.ID.NO.:2 and SEQ.ID.NO.:4 are found in two retina-specific ESTs deposited in GenBank by The Institute for Genomic Research (accession numbers AA318352 and AA317489). Other ESTSs that correspond to this cDNA are accession numbers AA307119 (from a colon carcinoma), AA205892 (from neuronal cell line), and AA326727 (from human cerebellum). A true mouse ortholog of the CG1CE gene is represented in the mouse EST AA497726 (from mouse testis).

The novel DNA sequences of the present invention encoding CG1CE, in whole or in part, can be linked with other DNA sequences, i.e., DNA sequences to which CG1CE is not naturally linked, to form “recombinant DNA molecules” encoding CG1CE. Such other sequences can include DNA sequences that control transcription or translation such as, e.g., translation initiation sequences, promoters for RNA polymerase II, transcription or translation termination sequences, enhancer sequences, sequences that control replication in microorganisms, sequences that confer antibiotic resistance, or sequences that encode a polypeptide “tag” such as, e.g., a polyhistidine tract or the myc epitope. The novel DNA sequences of the present invention can be inserted into vectors such as plasmids, cosmids, viral vectors, P1 artificial chromosomes, or yeast artificial chromosomes.

Included in the present invention are DNA sequences that hybridize to at least one of SEQ.ID.NOs.:1, 2, or 4 under stringent conditions. By way of example, and not limitation, a procedure using conditions of high stringency is as follows: Prehybridization of filters containing DNA is carried out for 2 hr. to overnight at 65° C. in buffer composed of 6×SSC, 5× Denhardt's solution, and 100 μg/ml denatured salmon sperm DNA. Filters are hybridized for 12 to 48 hrs at 65° C. in prehybridization mixture containing 100 μg/ml denatured salmon sperm DNA and 5-20×10⁶ cpm of ³²P-labeled probe. Washing of filters is done at 37° C. for 1 hr in a solution containing 2×SSC, 0.1% SDS. This is followed by a wash in 0.1×SSC, 0.1% SDS at 50° C. for 45 min. before autoradiography.

Other procedures using conditions of high stringency would include either a hybridization carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Reagents mentioned in the foregoing procedures for carrying out high stringency hybridization are well known in the art. Details of the composition of these reagents can be found in, e.g., Sambrook, Fritsch, and Maniatis, 1989, Molecular Cloning: A Laboratory Manual, second edition, Cold Spring Harbor Laboratory Press. In addition to the foregoing, other conditions of high stringency which may be used are well known in the art.

The degeneracy of the genetic code is such that, for all but two amino acids, more than a single codon encodes a particular amino acid. This allows for the construction of synthetic DNA that encodes the CG I CE protein where the nucleotide sequence of the synthetic DNA differs significantly from the nucleotide sequences of SEQ.ID.NOs.:2 or 4, but still encodes the same CG1CE protein as SEQ.ID.NOs.:2 or 4. Such synthetic DNAs are intended to be within the scope of the present invention.

Mutated forms of SEQ.ID.NOs.:1, 2, or 4 are intended to be within the scope of the present invention. In particular, mutated forms of SEQ.ID.NOs.:1, 2, or 4 which give rise to Best's macular dystrophy are within the scope of the present invention. Accordingly, the present invention includes a DNA molecule having a nucleotide sequence that is identical to SEQ.ID.NO.:1 except that the nucleotide at position 7,259 of SEQ.ID.NO.:1 is T, A, or C rather than G, so that the codon at positions 7,257-7,259 encodes either cysteine or is a stop codon rather than encoding tryptophan. Also included in the present invention is a DNA molecule having a nucleotide sequence that is identical to SEQ.ID.NO.:1 except that at least one of the nucleotides at position 7,257 or 7,258 has been changed so that the codon at positions 7,257-7,259 does not encode tryptophan.

The present invention includes a DNA molecule having a nucleotide sequence that is identical to positions 105-1,859 of SEQ.ID.NO.:2 except that the nucleotide at position 383 is T, A, or C rather than G, so that the codon at positions 381-383 encodes either cysteine or is a stop codon rather than encoding tryptophan. Also included in the present invention is a DNA molecule having a nucleotide sequence that is identical to positions 105-1,859 of SEQ.ID.NO.:2 except that at least one of the nucleotides at position 381 or 382 has been changed so that the codon at positions 381-383 does not encode tryptophan.

The present invention includes a DNA molecule having a nucleotide sequence that is identical to positions 105-1,409 of SEQ.ID.NO.:4 except that the nucleotide at position 383 is T, A, or C rather than G, so that the codon at positions 381-383 encodes either cysteine or is a stop codon rather than encoding tryptophan. Also included in the present invention is a DNA molecule having a nucleotide sequence that is identical to positions 105-1,409 of SEQ.ID.NO.:4 except that at least one of the nucleotides at position 381 or 382 has been changed so that the codon at positions 381-383 does not encode tryptophan.

The present invention includes a DNA molecule having a nucleotide sequence that is identical to SEQ.ID.NO.:1 except that the nucleotide at position 7,233 of SEQ.ID.NO.:1 is C, A, or G rather than T, so that the codon at positions 7,233-7,235 does not encode tyrosine. Also included in the present invention is a DNA molecule having a nucleotide sequence that is identical to SEQ.ID.NO.:1 except that at least one of the nucleotides at position 7,234 or 7,235 has been changed so that the codon at positions 7,233-7,235 does not encode tyrosine.

The present invention includes a DNA molecule having a nucleotide sequence that is identical to positions 105-1,859 of SEQ.ID.NO.:2 except that the nucleotide at position 357 is C, A, or G rather than T, so that the codon at positions 357-359 does not encode tyrosine. Also included in the present invention is a DNA molecule having a nucleotide sequence that is identical to positions 105-1,859 of SEQ.ID.NO.:2 except that at least one of the nucleotides at position 358 or 359 has been changed so that the codon at positions 357-359 does not encode tyrosine.

The present invention includes a DNA molecule having a nucleotide sequence that is identical to positions 105-1,409 of SEQ.ID.NO.:4 except that the nucleotide at position 357 is C, A, or G rather than T, so that the codon at positions 357-359 does not encode tyrosine. Also included in the present invention is a DNA molecule having a nucleotide sequence that is identical to positions 105-1,409 of SEQ.ID.NO.:4 except that at least one of the nucleotides at position 358 or 359 has been changed so that the codon at positions 357-359 does not encode tyrosine.

The present invention includes a DNA molecule having a nucleotide sequence that is identical to SEQ.ID.NO.:1 except that the nucleotide at position 3,330 is C rather than A. Also included in the present invention is a DNA molecule having a nucleotide sequence that is identical to SEQ.ID.NO.:1 except that the nucleotide at position 3,330 of SEQ.ID.NO.:1 is G, C, or T rather than A, so that the codon at positions 3,330-3,332 does not encode threonine. Also included in the present invention is a DNA molecule having a nucleotide sequence that is identical to SEQ.ID.NO.:1 except that at least one of the nucleotides at position 3,330 or 3,331 has been changed so that the codon at positions 3,330-3,332 does not encode threonine.

The present invention includes a DNA molecule having a nucleotide sequence that is identical to positions 105-1,859 of SEQ.ID.NO.:2 except that the nucleotide at position 120 is C rather than A. Also included in the present invention is a DNA molecule having a nucleotide sequence that is identical to positions 105-1,859 of SEQ.ID.NO.:2 except that the nucleotide at position 120 is G, C, or T rather than A, so that the codon at positions 120-122 does not encode threonine. Also included in the present invention is a DNA molecule having a nucleotide sequence that is identical to positions 105-1,859 of SEQ.ID.NO.:2 except that at least one of the nucleotides at position 120 or 121 has been changed so that the codon at positions 120-122 does not encode threonine.

The present invention includes a DNA molecule having a nucleotide sequence that is identical to positions 105-1,409 of SEQ.ID.NO.:4 except that the nucleotide at position 120 is C rather than A. Also included in the present invention is a DNA molecule having a nucleotide sequence that is identical to positions 105-1,409 of SEQ.ID.NO.:4 except that the nucleotide at position 120 is G, C, or T rather than A, so that the codon at positions 120-122 does not encode threonine. Also included in the present invention is a DNA molecule having a nucleotide sequence that is identical to positions 105-1,409 of SEQ.ID.NO.:4 except that at least one of the nucleotides at position 120 or 121 has been changed so that the codon at positions 120-122 does not encode threonine.

The present invention includes a DNA molecule having a nucleotide sequence that is identical to SEQ.ID.NO.:1 except that the nucleotide at position 8,939 is A rather than T. Also included in the present invention is a DNA molecule having a nucleotide sequence that is identical to SEQ.ID.NO.:1 except that the nucleotide at position 8,939 of SEQ.ID.NO.:1 is A, G, or C, rather than T, so that the codon at positions 8,939-8,941 does not encode tyrosine. Also included in the present invention is a DNA molecule having a nucleotide sequence that is identical to SEQ.ID.NO.:1 except that at least one of the nucleotides at position 8,939-8,941 has been changed so that the codon at positions 8,939-8,941 does not encode tyrosine.

The present invention includes a DNA molecule having a nucleotide sequence that is identical to positions 105-1,859 of SEQ.ID.NO.:2 except that the nucleotide at position 783 is A rather than T, Also included in the present invention is a DNA molecule having a nucleotide sequence that is identical to positions 105-1,859 of SEQ.ID.NO.:2 except that the nucleotide at position 783 is A, G, or C rather than T so that the codon at positions 783-785 does not encode tyrosine. Also included in the present invention is a DNA molecule having a nucleotide sequence that is identical to positions 105-1,859 of SEQ.ID.NO.:2 except that at least one of the nucleotides at position 783-785 has been changed so that the codon at positions 783-785 does not encode tyrosine.

The present invention includes a DNA molecule having a nucleotide sequence that is identical to positions 105-1,409 of SEQ.ID.NO.:4 except that the nucleotide at position 783 is A rather than T. Also included in the present invention is a DNA molecule having a nucleotide sequence that is identical to positions 105-1,409 of SEQ.ID.NO.:4 except that the nucleotide at position 783 is A, G, or C rather than T, so that the codon at positions 783-785 does not encode tyrosine. Also included in the present invention is a DNA molecule having a nucleotide sequence that is identical to positions 105-1,409 of SEQ.ID.NO.:4 except that at least one of the nucleotides at position 783-785 has been changed so that the codon at positions 783-785 does not encode tyrosine.

The present invention includes a DNA molecule having a nucleotide sequence that is identical to SEQ.ID.NO.:1 except that the nucleotide at position 11,241 is A rather than G. Also included in the present invention is a DNA molecule having a nucleotide sequence that is identical to SEQ.ID.NO.:1 except that the nucleotide at position 11,241 is A, C, or T, rather than G, so that the codon at positions 11,240-11,242 does not encode glycine. Also included in the present invention is a DNA molecule having a nucleotide sequence that is identical to SEQ.ID.NO.:1 except that at least one of the nucleotides at position 11,240 or 11,241 has been changed so that the codon at positions 11,240-11,242 does not encode glycine.

The present invention includes a DNA molecule having a nucleotide sequence that is identical to positions 105-1,859 of SEQ.ID.NO.:2 except that the nucleotide at position 1,000 is A rather than G. Also included in the present invention is a DNA molecule having a nucleotide sequence that is identical to positions 105-1,859 of SEQ.ID.NO.:2 except that the nucleotide at position 1,000 is A, C, or T rather than G, so that the codon at positions 999-1,001 does not encode glycine. Also included in the present invention is a DNA molecule having a nucleotide sequence that is identical to positions 105-1,859 of SEQ.ID.NO.:2 except that at least one of the nucleotides at position 999 or 1,000 has been changed so that the codon at positions 999-1,001 does not encode glycine.

Another aspect of the present invention includes host cells that have been engineered to contain and/or express DNA sequences encoding CG1CE protein. Such recombinant host cells can be cultured under suitable conditions to produce CG1CE protein. An expression vector containing DNA encoding CG1CE protein can be used for expression of CG1CE protein in a recombinant host cell. Recombinant host cells may be prokaryotic or eukaryotic, including but not limited to, bacteria such as E. coli, fungal cells such as yeast, mammalian cells including, but not limited to, cell lines of human, bovine, porcine, monkey and rodent origin, and insect cells including but not limited to Drosophila and silkworm derived cell lines. Cell lines derived from mammalian species which are suitable for recombinant expression of CG1CE protein and which are commercially available, include but are not limited to, L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) and MRC-5 (ATCC CCL 171).

A variety of mammalian expression vectors can be used to express recombinant CG1CE in mammalian cells. Commercially available mammalian expression vectors which are suitable include, but are not limited to, pMClneo (Stratagene), pSG5 (Stratagene), pcDNAI and pcDNAIamp, pcDNA3, pcDNA3.1, pCR3.1 (Invitrogen), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), and pSV2-dhfr (ATCC 37146). Following expression in recombinant cells, CG1CE can be purified by conventional techniques to a level that is substantially free from other proteins.

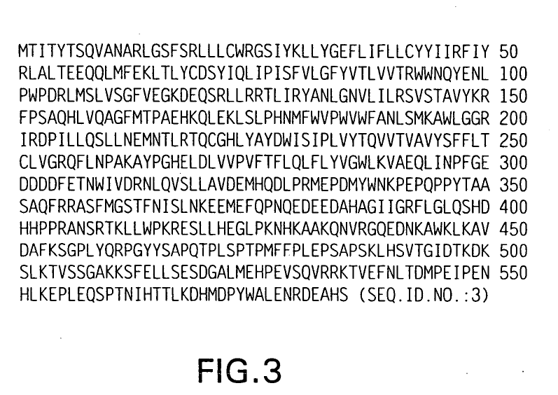

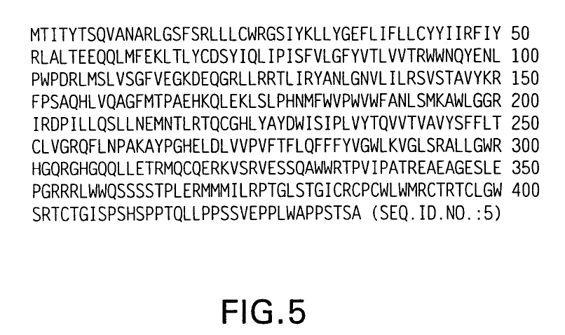

The present invention includes CG1CE protein substantially free from other proteins. The amino acid sequence of the full-length CG1CE protein is shown in FIG. 3 as SEQ.ID.NO.:3. Thus, the present invention includes CG1CE protein substantially free from other proteins having the amino acid sequence SEQ.ID.NO.:3. Also included in the present invention is a CG1CE protein that is produced from an alternatively spliced CG1CE mRNA where the protein has the amino acid sequence shown in FIG. 5 as SEQ.ID.NO.:5.

Mutated forms of CG1CE proteins are intended to be within the scope of the present invention. In particular, mutated forms of SEQ.ID.NOs.:3 and 5 that give rise to Best's macular dystrophy are within the scope of the present invention. Accordingly, the present invention includes a protein having the amino acid sequence shown in FIG. 3 as SEQ.ID.NO.:3 except that the amino acid at position 93 is cysteine rather than tryptophan. The present invention also includes a protein having the amino acid sequence shown in FIG. 5 as SEQ.ID.NO.:5 except that the amino acid at position 93 is cysteine rather than tryptophan. The present invention includes a protein having the amino acid sequence shown in FIG. 3 as SEQ.ID.NO.:3 except that the amino acid at position 93 is not tryptophan. The present invention also includes a protein having the amino acid sequence shown in FIG. 5 as SEQ.ID.NO.:5 except that the amino acid at position 93 is not tryptophan.

The present invention includes a protein having the amino acid sequence shown in FIG. 3 as SEQ.ID.NO.:3 except that the amino acid at position 85 is histidine rather than tyrosine. The present invention also includes a protein having the amino acid sequence shown in FIG. 5 as SEQ.ID.NO.:5 except that the amino acid at position 85 is histidine rather than tyrosine. The present invention includes a protein having the amino acid sequence shown in FIG. 3 as SEQ.ID.NO.:3 except that the amino acid at position 85 is not tyrosine. The present invention also includes a protein having the amino acid sequence shown in FIG. 5 as SEQ.ID.NO.:5 except that the amino acid at position 85 is not tyrosine.

The present invention includes a protein having the amino acid sequence shown in FIG. 3 as SEQ.ID.NO.:3 except that the amino acid at position 6 is proline rather than threonine. The present invention also includes a protein having the amino acid sequence shown in FIG. 5 as SEQ.ID.NO.:5 except that the amino acid at position 6 is proline rather than threonine. The present invention includes a protein having the amino acid sequence shown in FIG. 3 as SEQ.ID.NO.:3 except that the amino acid at position 6 is not threonine. The present invention also includes a protein having the amino acid sequence shown in FIG. 5 as SEQ.ID.NO.:5 except that the amino acid at position 6 is not threonine.

The present invention includes a protein having the amino acid sequence shown in FIG. 3 as SEQ.ID.NO.:3 except that the amino acid at position 227 is asparagine rather than tyrosine. The present invention also includes a protein having the amino acid sequence shown in FIG. 5 as SEQ.ID.NO.:5 except that the amino acid at position 227 is asparagine rather than tyrosine. The present invention includes a protein having the amino acid sequence shown in FIG. 3 as SEQ.ID.NO.:3 except that the amino acid at position 227 is not tyrosine. The present invention also includes a protein having the amino acid sequence shown in FIG. 5 as SEQ.ID.NO.:5 except that the amino acid at position 227 is not tyrosine.

The present invention includes a protein having the amino acid sequence shown in FIG. 3 as SEQ.ID.NO.:3 except that the amino acid at position 299 is glutamate rather than glycine. The present invention includes a protein having the amino acid sequence shown in FIG. 3 as SEQ.ID.NO.:3 except that the amino acid at position 299 is not glycine.

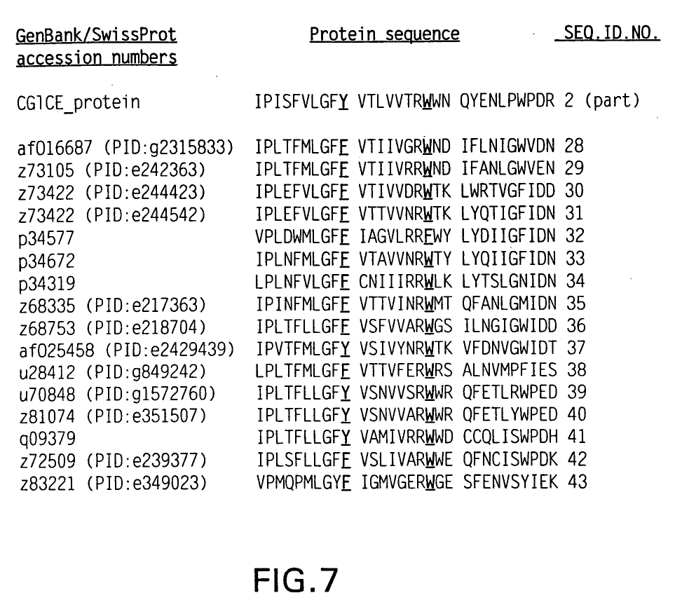

As with many proteins, it is possible to modify many of the amino acids of CG1CE and still retain substantially the same biological activity as the original protein. Thus, the present invention includes modified CG1CE proteins which have amino acid deletions, additions, or substitutions but that still retain substantially the same biological activity as CG1CE. It is generally accepted that single amino acid substitutions do not usually alter the biological activity of a protein (see, e.g., Molecular Biology of the Gene, Watson et al., 1987, Fourth Ed., The Benjamin/Cummings Publishing Co., Inc., page 226; and Cunningham & Wells, 1989, Science 244:1081-1085). Accordingly, the present invention includes polypeptides where one amino acid substitution has been made in SEQ.ID.NOs.:3 or 5 wherein the polypeptides still retain substantially the same biological activity as CG1CE. The present invention also includes polypeptides where two amino acid substitutions have been made in SEQ.ID.NOs.:3 or 5 wherein the polypeptides still retain substantially the same biological activity as CG1CE. In particular, the present invention includes embodiments where the above-described substitutions are conservative substitutions. In particular, the present invention includes embodiments where the above-described substitutions do not occur in positions where the amino acid present in CG1CE is also present in one of the C. elegans proteins whose partial sequence is shown in FIG. 7.

The CG1CE proteins of the present invention may contain post-translational modifications, e.g., covalently linked carbohydrate.

The present invention also includes chimeric CG1CE proteins. Chimeric CG1CE proteins consist of a contiguous polypeptide sequence of at least a portion of a CG1CE protein fused to a polypeptide sequence of a non-CG1CE protein.

The present invention also includes isolated forms of CG1CE proteins and CG1CE DNA. By “isolated CG1CE protein” or “isolated CG1CE DNA” is meant CG1CE protein or DNA encoding CG1CE protein that has been isolated from a natural source. Use of the term “isolated” indicates that CG1CE protein or CG1CE DNA has been removed from its normal cellular environment. Thus, an isolated CG1CE protein may be in a cell-free solution or placed in a different cellular environment from that in which it occurs naturally. The term isolated does not imply that an isolated CG1CE protein is the only protein present. but instead means that an isolated CG1CE protein is at least 95% free of non-amino acid material (e.g., nucleic acids, lipids, carbohydrates) naturally associated with the CG1CE protein. Thus, a CG1CE protein that is expressed in bacteria or even in eukaryotic cells which do not naturally (i.e., without human intervention) express it through recombinant means is an “isolated CG1CE protein.”

A cDNA fragment encoding full-length CG1CE can be isolated from a human retinal cell cDNA library by using the polymerase chain reaction (PCR) employing suitable primer pairs. Such primer pairs can be selected based upon the cDNA sequence for CG1CE shown in FIG. 2 as SEQ.ID.NO.:2 or in FIG. 4 as SEQ.ID.NO.:4. Suitable primer pairs would be, e.g.:

| | |
|---|---|
| CAGGGAGTCCCACCAGCC | (SEQ.ID.NO.:6) |
| and | |
| TCCCCATTAGGAAGCAGG | (SEQ.ID.NO.:7) |
| for SEQ.ID.NO.:2; | |
| and | |
| CAGGGAGTCCCACCAGCC | (SEQ.ID.NO.:6) |
| and | |
| TCTCCTCTTTGTTCAGGC | (SEQ.ID.NO.:8) |
| for SEQ.ID.NO.:4. | |

PCR reactions can be carried out with a variety of thermostable enzymes including but not limited to AmpliTaq, AmpliTaq Gold, or Vent polymerase. For AmpliTaq, reactions can be carried out in 10 mM Tris-Cl, pH 8.3, 2.0 mM MgCl₂, 200 μM for each dNTP, 50 mM KCl, 0.2 μM for each primer, 10 ng of DNA template, 0.05 units/μl of AmpliTaq. The reactions are heated at 95° C. for 3 minutes and then cycled 35 times using the cycling parameters of 95° C., 20 seconds, 62° C., 20 seconds, 72° C., 3 minutes. In addition to these conditions, a variety of suitable PCR protocols can be found in PCR Primer, A Laboratory Manual, edited by C. W. Dieffenbach and G. S. Dveksler, 1995, Cold Spring Harbor Laboratory Press; or PCR Protocols: A Guide to Methods and Applications, Michael et al., eds., 1990, Academic Press.

A suitable cDNA library from which a clone encoding CG1CE can be isolated would be Human Retina 5′-stretch cDNA library in lambda gt10 or lambda gt11 vectors (catalog numbers HL1143a and HL1132b, Clontech, Palo Alto, Calif.). The primary clones of such a library can be subdivided into pools with each pool containing approximately 20,000 clones and each pool can be amplified separately.

By this method, a cDNA fragment encoding an open reading frame of 585 amino acids (SEQ.ID.NO.:3) or an open reading frame of 435 amino acids (SEQ.ID.NO.:5) can be obtained. This cDNA fragment can be cloned into a suitable cloning vector or expression vector. For example, the fragment can be cloned into the mammalian expression vector pcDNA3.1 (Invitrogen, San Diego, Calif.). CG I CE protein can then be produced by transferring an expression vector encoding CG1CE or portions thereof into a suitable host cell and growing the host cell under appropriate conditions. CG1CE protein can then be isolated by methods well known in the art.

As an alternative to the above-described PCR method, a cDNA clone encoding CG1CE can be isolated from a cDNA library using as a probe oligonucleotides specific for CG1CE and methods well known in the art for screening cDNA libraries with oligonucleotide probes. Such methods are described in, e.g., Sambrook et al., 1989, Molecular Cloning: A Laboratory Manual; Cold Spring Harbor Laboratory, Cold Spring Harbor, N.Y.; Glover, D. M. (ed.), 1985, DNA Cloning: A Practical Approach, MRL Press, Ltd., Oxford, U.K., Vol. I, II. Oligonucleotides that are specific for CG1CE and that can be used to screen cDNA libraries can be readily designed based upon the cDNA sequence of CG1CE shown in FIG. 2 as SEQ.ID.NO.:2 or in FIG. 4 as SEQ.ID.NO.:4 and can be synthesized by methods well-known in the art.

Genomic clones containing the CG1CE gene can be obtained from commercially available human PAC or BAC libraries available from Research Genetics, Huntsville, Ala. PAC clones containing the CG1CE gene (e.g., PAC 759J12, PAC 466A11) are commercially available from Research Genetics, Huntsville, Ala. (Catalog number for individual PAC clones is RPCI.C). Alternatively, one may prepare genomic libraries, especially in P1 artificial chromosome vectors, from which genomic clones containing the CG1CE can be isolated, using probes based upon the CG1CE sequences disclosed herein. Methods of preparing such libraries are known in the art (Ioannou et al., 1994, Nature Genet. 6:84-89).

The novel DNA sequences of the present invention can be used in various diagnostic methods relating to Best's macular dystrophy. The present invention provides diagnostic methods for determining whether a patient carries a mutation in the CG1CE gene that predisposes that patient toward the development of Best's macular dystrophy. In broad terms, such methods comprise determining the DNA sequence of a region of the CG1CE gene from the patient and comparing that sequence to the sequence from the corresponding region of the CG1CE gene from a normal person, i.e., a person who does not suffer from Best's macular dystrophy.

Such methods of diagnosis may be carried out in a variety of ways. For example, one embodiment comprises:

(a) providing PCR primers from a region of the CG1CE gene where it is suspected that a patient harbors a mutation in the CG1CE gene;

(b) performing PCR on a DNA sample from the patient to produce a PCR fragment from the patient;

(c) performing PCR on a control DNA sample having a nucleotide sequence selected from the group consisting of SEQ.ID.NOs.:1, 2 and SEQ.ID.NO.:4 to produce a control PCR fragment;

(d) determining the nucleotide sequence of the PCR fragment from the patient and the nucleotide sequence of the control PCR fragment;

(e) comparing the nucleotide sequence of the PCR fragment from the patient to the nucleotide sequence of the control PCR fragment;

where a difference between the nucleotide sequence of the PCR fragment from the patient and the nucleotide sequence of the control PCR fragment indicates that the patient has a mutation in the CG1CE gene.

In a particular embodiment, the PCR primers are from the coding region of the CG1CE gene, i.e., from the coding region of SEQ.ID.NOs.:1, 2, or 4.

In a particular embodiment, the DNA sample from the patient is cDNA that has been prepared from an RNA sample from the patient. In another embodiment, the DNA sample from the patient is genomic DNA.

In a particular embodiment, the nucleotide sequences of the PCR fragment from the patient and the control PCR fragment are determined by DNA sequencing.

In a particular embodiment, the nucleotide sequences of the PCR fragment from the patient and the control PCR fragment are compared by direct comparison after DNA sequencing. In another embodiment, the comparison is made by a process that includes hybridizing the PCR fragment from the patient and the control PCR fragment and then using an endonuclease that cleaves at any mismatched positions in the hybrid but does not cleave the hybrid if the two fragments match perfectly. Such an endonuclease is, e.g., S1. In this embodiment, the conversion of the PCR fragment from the patient to smaller fragments after endonuclease treatment indicates that the patient carries a mutation in the CG1CE gene. In such embodiments, it may be advantageous to label (radioactively, enzymatically, immunologically, etc.) the PCR fragment from the patient or the control PCR fragment.

The present invention provides a method of diagnosing whether a patient carries a mutation in the CG1CE gene that comprises:

(a) obtaining an RNA sample from the patient;

(b) performing reverse transcription-PCR (RT-PCR) on the RNA sample using primers that span a region of the coding sequence of the CG1CE gene to produce a PCR fragment from the patient where the PCR fragment from the patient has a defined length, the length being dependent upon the identity of the primers that were used in the RT-PCR;

(c) hybridizing the PCR fragment to DNA having a sequence selected from the group consisting of SEQ.ID.NOs.:1, 2 and SEQ.ID.NO.:4 to form a hybrid;

(d) treating the hybrid produced in step (c) with an endonuclease that cleaves at any mismatched positions in the hybrid but does not cleave the hybrid if the two fragments match perfectly;

(e) determining whether the endonuclease cleaved the hybrid by determining the length of the PCR fragment from the patient after endonuclease treatment where a reduction in the length of the PCR fragment from the patient after endonuclease treatment indicates that the patient carries a mutation in the CG1CE gene.

The present invention provides a method of diagnosing whether a patient carries a mutation in the CG1CE gene that comprises:

(a) making cDNA from an RNA sample from the patient;

(b) providing a set of PCR primers based upon SEQ.ID.NO.:2 or SEQ.ID.NO.:4;

(c) performing PCR on the cDNA to produce a PCR fragment from the patient;

(d) determining the nucleotide sequence of the PCR fragment from the patient;

(e) comparing the nucleotide sequence of the PCR fragment from the patient with the nucleotide sequence of SEQ.ID.NO.:2 or SEQ.ID.NO.:4;

where a difference between the nucleotide sequence of the PCR fragment from the patient with the nucleotide sequence of SEQ.ID.NO.:2 or SEQ.ID.NO.:4 indicates that the patient carries a mutation in the CG1CE gene.

The present invention provides a method of diagnosing whether a patient carries a mutation in the CG1CE gene that comprises:

(a) preparing genomic DNA from the patient;

(b) providing a set of PCR primers based upon SEQ.ID.NO.:1, SEQ.ID.NO.:2, or SEQ.ID.NO.:4;

(c) performing PCR on the genomic DNA to produce a PCR fragment from the patient;

(d) determining the nucleotide sequence of the PCR fragment from the patient;

(e) comparing the nucleotide sequence of the PCR fragment from the patient with the nucleotide sequence of SEQ.ID.NO.:2 or SEQ.ID.NO.:4;

where a difference between the nucleotide sequence of the PCR fragment from the patient with the nucleotide sequence of SEQ.ID.NO.:2 or SEQ.ID.NO.:4 indicates that the patient carries a mutation in the CG1CE gene.

In a particular embodiment, the primers are selected so that they amplify a portion of SEQ.ID.NOs.:2 or 4 that includes at least one position selected from the group consisting of: positions 120, 121, 122, 357, 358, 359, 381, 382, 383, 783, 784, and 785. In another embodiment, the primers are selected so that they amplify a portion of SEQ.ID.NOs.:2 or 4 that includes at least one position selected from the group consisting of: positions 384, 385, and 386. In another embodiment, the primers are selected so that they amplify a portion of SEQ.ID.NO.:2 that includes at least one position selected from the group consisting of: positions 999, 1,000, and 1,001. In another embodiment, the primers are selected so that they amplify a portion of SEQ.ID.NOs.:2 or 4 that includes at least one codon that encodes an amino acid present in CG1CE that is also present in the corresponding position in at least one of the C. elegans proteins whose partial amino acid sequence is shown in FIG. 7.

In a particular embodiment, the present invention provides a diagnostic method for determining whether a person carries a mutation of the CG1CE gene in which the G at position 383 of SEQ.ID.NO.:2 has been changed to a C. This change results in the creation of a Fnu4HI restriction site. By amplifying a PCR fragment spanning position 383 of SEQ.ID.NO.:2 from DNA or cDNA prepared from a person, digesting the PCR fragment with Fnu4HI, and visualizing the digestion products, e.g., by SDS-PAGE, one can easily determine if the person carries the G383C mutation. For example, one could use the PCR primer pair 5′-CTCCTGCCCAGGCTTCTAC-3′ (SEQ.ID.NO.:30) and 5′-CTTGCTCTGCCTTGCCTTC-3′ (SEQ.ID.NO.:31) to amplify a 125 base pair fragment. Heterozygotes for the G383C mutation have three Fnu4HI digestion products: 125 bp, 85 bp, and 40 bp; homozygotes have two: 85 bp and 40 bp; and wild-type individuals have a single fragment of 125 bp.

In a particular embodiment, the present invention provides a diagnostic method for determining whether a person carries a mutation of the CG1CE gene in which the T at position 783 of SEQ.ID.NO.:2 has been changed to an A. This change results in the creation of a PflMI restriction site. By amplifying a PCR fragment spanning position 783 of SEQ.ID.NO.:2 from DNA or cDNA prepared from a person, digesting the PCR fragment with PflMI, and visualizing the digestion products, e.g., by SDS-PAGE, one can easily determine if the person carries the T783A mutation.

The present invention also provides oligonucleotide probes, based upon the sequences of SEQ.ID.NOs.:1, 2, or 4, that can be used in diagnostic methods related to Best's macular dystrophy. In particular, the present invention includes DNA oligonucleotides comprising at least 18 contiguous nucleotides of at least one of a sequence selected from the group consisting of: SEQ.ID.NOs.:1, 2 and SEQ.ID.:NO.4. Also provided by the present invention are corresponding RNA oligonucleotides. The DNA or RNA oligonucleotide probes can be packaged in kits.

In addition to the diagnostic utilities described above, the present invention makes possible the recombinant expression of the CG1CE protein in various cell types. Such recombinant expression makes possible the study of this protein so that its biochemical activity and its role in Best's macular dystrophy can be elucidated.

The present invention also makes possible the development of assays which measure the biological activity of the CG1CE protein. Such assays using recombinantly expressed CG1CE protein are especially of interest. Assays for CG1CE protein activity can be used to screen libraries of compounds or other sources of compounds to identify compounds that are activators or inhibitors of the activity of CG1CE protein. Such identified compounds can serve as “leads” for the development of pharmaceuticals that can be used to treat patients having Best's macular dystrophy. In versions of the above-described assays, mutant CG1CE proteins are used and inhibitors or activators of the activity of the mutant CG1CE proteins are discovered.

Such assays comprise:

(a) recombinantly expressing CG1CE protein or mutant CG1CE protein in a host cell;

(b) measuring the biological activity of CG1CE protein or mutant CG1CE protein in the presence and in the absence of a substance suspected of being an activator or an inhibitor of CG1CE protein or mutant CG1CE protein;

where a change in the biological activity of the CG1CE protein or the mutant CG1CE protein in the presence as compared to the absence of the substance indicates that the substance is an activator or an inhibitor of CG1CE protein or mutant CG1CE protein.

The present invention also includes antibodies to the CG1CE protein. Such antibodies may be polyclonal antibodies or monoclonal antibodies. The antibodies of the present invention are raised against the entire CG1CE protein or against suitable antigenic fragments of the protein that are coupled to suitable carriers, e.g., serum albumin or keyhole limpet hemocyanin, by methods well known in the art. Methods of identifying suitable antigenic fragments of a protein are known in the art. See, e.g., Hopp & Woods, 1981, Proc. Natl. Acad. Sci. USA 78:3824-3828; and Jameson & Wolf, 1988, CABIOS (Computer Applications in the Biosciences) 4:181-186.

For the production of polyclonal antibodies, CG1CE protein or an antigenic fragment, coupled to a suitable carrier, is injected on a periodic basis into an appropriate non-human host animal such as, e.g., rabbits, sheep, goats, rats, mice. The animals are bled periodically and sera obtained are tested for the presence of antibodies to the injected antigen. The injections can be intramuscular, intraperitoneal, subcutaneous, and the like, and can be accompanied with adjuvant.

For the production of monoclonal antibodies, CG1CE protein or an antigenic fragment, coupled to a suitable carrier, is injected into an appropriate non-human host animal as above for the production of polyclonal antibodies. In the case of monoclonal antibodies, the animal is generally a mouse. The animal's spleen cells are then immortalized, often by fusion with a myeloma cell, as described in Kohler & Milstein, 1975, Nature 256:495-497. For a fuller description of the production of monoclonal antibodies, see Antibodies: A Laboratory Manual, Harlow & Lane, eds., Cold Spring Harbor Laboratory Press, 1988.

Gene therapy may be used to introduce CG1CE polypeptides into the cells of target organs, e.g., the pigmented epithelium of the retina or other parts of the retina. Nucleotides encoding CG1CE polypeptides can be ligated into viral vectors which mediate transfer of the nucleotides by infection of recipient cells. Suitable viral vectors include retrovirus, adenovirus, adeno-associated virus, herpes virus, vaccinia virus, and polio virus based vectors. Alternatively, nucleotides encoding CG1CE polypeptides can be transferred into cells for gene therapy by non-viral techniques including receptor-mediated targeted transfer using ligand-nucleotide conjugates, lipofection, membrane fusion, or direct microinjection. These procedures and variations thereof are suitable for ex vivo as well as in vivo gene therapy. Gene therapy with CG1CE polypeptides will be particularly useful for the treatment of diseases where it is beneficial to elevate CG1CE activity.

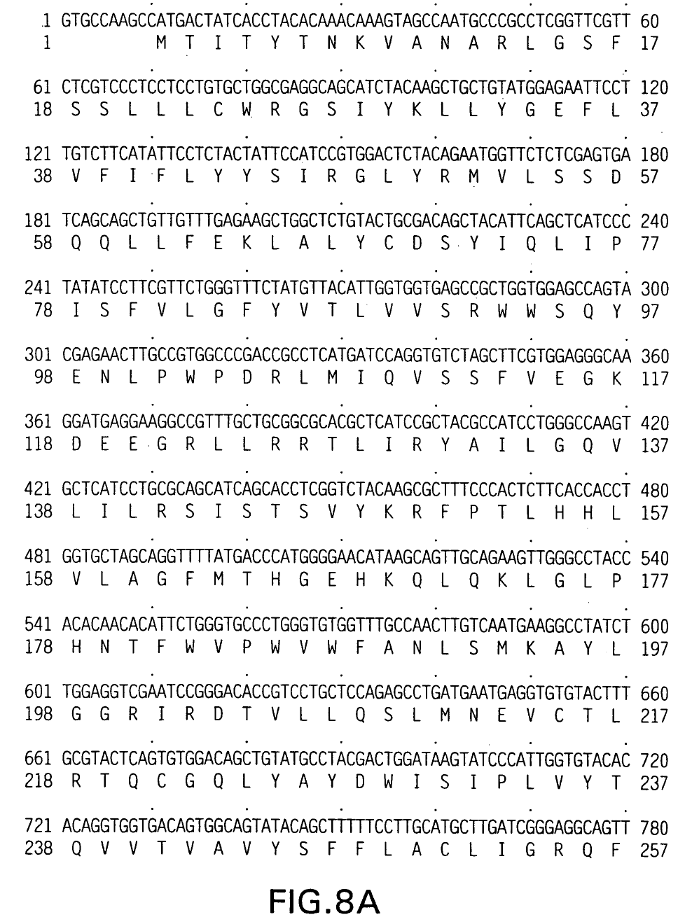

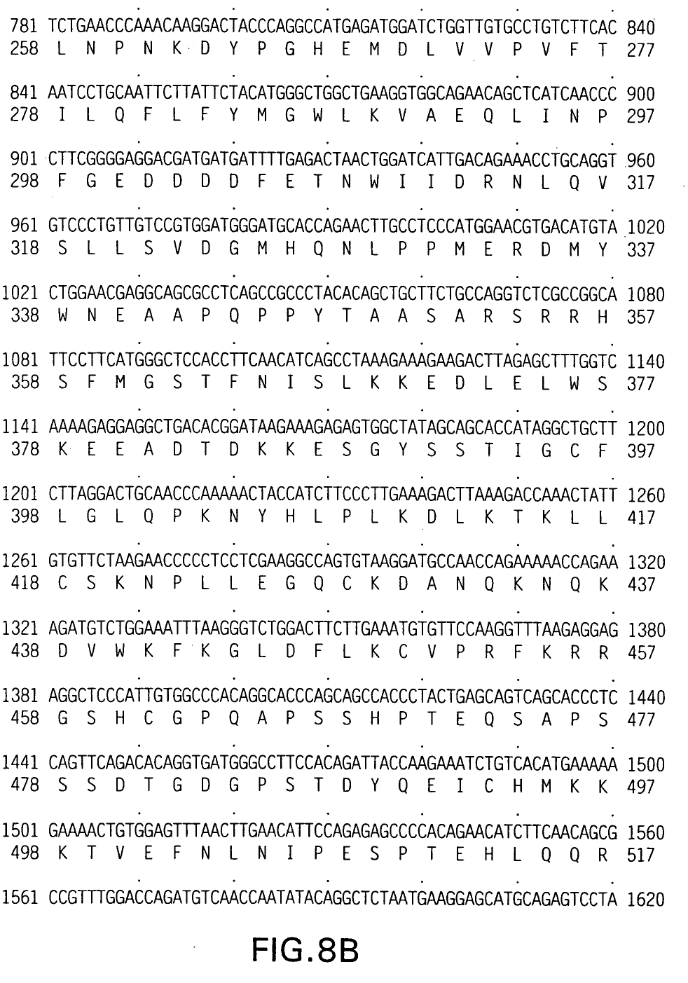

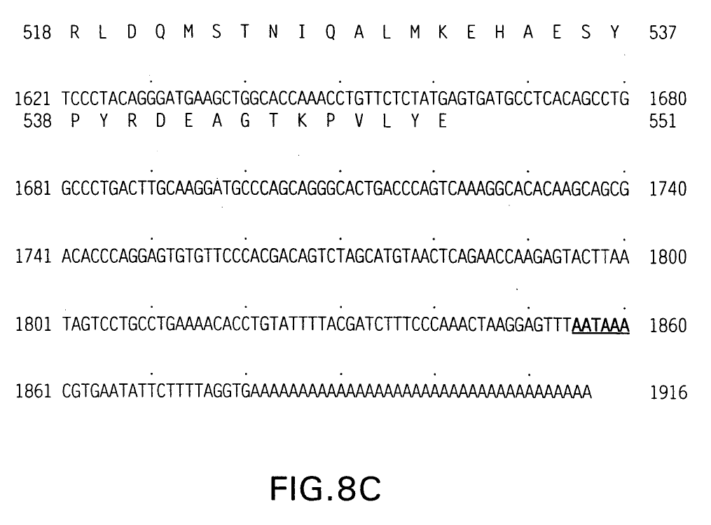

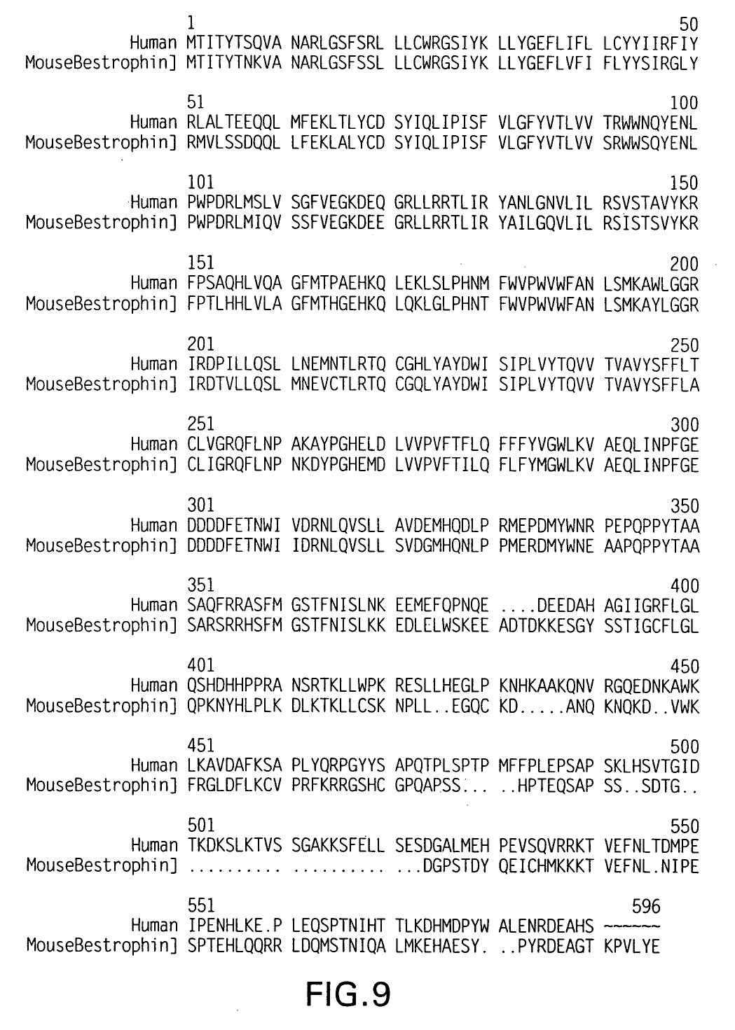

The present invention includes DNA comprising nucleotides encoding mouse CG1CE. Included within such DNA is the DNA sequence shown in FIG. 8A-C (SEQ. ID. NO.:28). Also included is DNA comprising positions 11-1,663 of SEQ. ID. NO.:28. Also included are mutant versions of DNA encoding mouse CG1CE. Included is DNA comprising nucleotides that are identical to positions 11-1,663 of SEQ. ID. NO.:28 except that at least one of the nucleotides at positions 26-28, positions 263-265, positions 287-289, positions 689-691, and/or positions 905-907 differs from the corresponding nucleotide at positions 26-28, positions 263-265, positions 287-289, positions 689-691, and/or positions 905-907 of SEQ. ID. NO.:28. Particularly preferred versions of mutant DNAs are those in which the nucleotide change results in a change in the corresponding encoded amino acid. The DNA encoding mouse CG1CE can be in isolated form, can be substantially free from other nucleic acids, and/or can be recombinant DNA.

The present invention includes mouse CG1CE protein (SEQ. ID. NO.:29). This mouse CG1CE protein can be in isolated form and/or can be sustantially free from other proteins. Mutant versions of mouse CG1CE protein are also part of the present invention. Examples of such mutant mouse CG1CE proteins are proteins that are identical to SEQ. ID. NO.:29 except that the amino acid at position 6, position 85, position 93, position 227, and/or position 299 differs from the corresponding amino acid at position 6, position 85, position 93, position 227, and/or position 299 in SEQ. ID. NO.:29.

cDNA encoding mouse CG1CE can be amplified by PCR from cDNA libraries made from mouse eye or mouse testis. Suitable primers can be readily designed based upon SEQ. ID. NO.:28. Alternatively, cDNA encoding mouse CG1CE can be isolated from cDNA libraries made from mouse eye or mouse testis by the use of oligonucleotide probes based upon SEQ. ID. NO.:28.

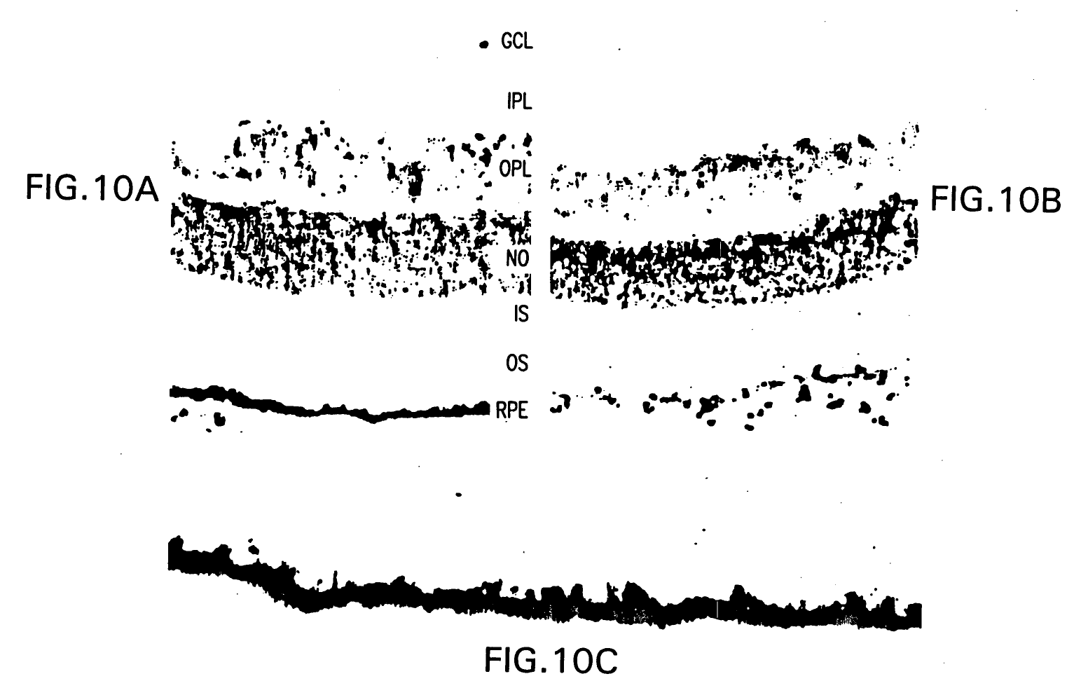

In situ hybridization studies demonstrated that mouse CG1CE is specifically expressed in the retinal pigmented epithelium (see FIG. 10).

By providing DNA encoding mouse CG1CE, the present invention allows for the generation of an animal model of Best's macular dystrophy. This animal model can be generated by making “knockout” or “knockin” mice containing altered CG1CE genes. Knockout mice can be generated in which portions of the mouse CG1CE gene have been deleted. Knockin mice can be generated in which mutations that have been shown to lead to Best's macular dystrophy when present in the human CG1CE gene are introduced into the mouse gene. In particular, mutations resulting in changes in amino acids 6, 85, 93, 227, or 299 of the mouse CG1CE protein (SEQ.ID.NO.:29) are contemplated. Such knockout and knockin mice will be valuable tools in the study of the Best's macular dystrophy disease process and will provide important model systems in which to test potential pharmaceuticals or treatments for Best's macular dystrophy.

Methods of producing knockout and knockin mice are well known in the art. For example, the use of gene-targeted ES cells in the generation of gene-targeted transgenic knockout mice is described in, e.g., Thomas et al., 1987, Cell 51:503-512, and is reviewed elsewhere (Frohman et al., 1989, Cell 56:145-147; Capecchi, 1989, Trends in Genet. 5:70-76; Baribault et al., 1989, Mol. Biol. Med. 6:481-492).

Techniques are available to inactivate or alter any genetic region to virtually any mutation desired by using targeted homologous recombination to insert specific changes into chromosomal genes. Generally, use is made of a “targeting vector,” i.e., a plasmid containing part of the genetic region it is desired to mutate. By virtue of the homology between this part of the genetic region on the plasmid and the corresponding genetic region on the chromosome, homologous recombination can be used to insert the plasmid into the genetic region, thus disrupting the genetic region. Usually, the targeting vector contains a selectable marker gene as well.

In comparison with homologous extrachromosomal recombination, which occurs at frequencies approaching 100%, homologous plasmid-chromosome recombination was originally reported to only be detected at frequencies between 10⁻⁶ and 10⁻³ (Lin et al., 1985, Proc. Natl. Acad. Sci. USA 82:1391-1395; Smithies et al., 1985, Nature 317: 230-234; Thomas et al., 1986, Cell 44:419-428). Nonhomologous plasmid-chromosome interactions are more frequent, occurring at levels 10⁵-fold (Lin et al., 1985, Proc. Natl. Acad. Sci. USA 82:1391-1395) to 10²-fold (Thomas et al., 1986, Cell 44:419-428) greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening individual clones (Kim et al., 1988, Nucleic Acids Res. 16:8887-8903; Kim et al., 1991, Gene 103:227-233). Alternatively, a positive genetic selection approach has been developed in which a marker gene is constructed which will only be active if homologous insertion occurs, allowing these recombinants to be selected directly (Sedivy et al., 1989, Proc. Natl. Acad. Sci. USA 86:227-231). One of the most powerful approaches developed for selecting homologous recombinants is the positive-negative selection (PNS) method developed for genes for which no direct selection of the alteration exists (Mansour et al., 1988, Nature 336:348-352; Capecchi, 1989, Science 244:1288-1292; Capecchi, 1989, Trends in Genet. 5:70-76). The PNS method is more efficient for targeting genes which are not expressed at high levels because the marker gene has its own promoter. Nonhomologous recombinants are selected against by using the Herpes Simplex virus thymidine kinase (HSV-TK) gene and selecting against its nonhomologous insertion with herpes drugs such as gancyclovir (GANC) or FIAU (1-(2-deoxy 2-fluoro-B-D-arabinofluranosyl)-5-iodouracil). By this counter-selection, the percentage of homologous recombinants in the surviving transformants can be increased.

The following non-limiting examples are presented to better illustrate the invention.

### EXAMPLE 1

Identification of the Human CG1CE Gene and cDNA Cloning

Construction of Libraries for Shotgun Sequencing

Bacterial strains containing the BMD PACs (P1 Artificial Chromosomes) were received from Research Genetics (Huntsville, Ala.). The minimum tiling path between markers D11S4076 and UGB that represents the minimum genetic region containing the BMD gene includes the following nine PAC clones: 363M5 (140 kb), 519O13 (120 kb), 527E4 (150 kb), 688P12 (140 kb), 741N15 (170 kb), 756B9 (120 kb), 759J12 (140 kb), 1079D9 (170 kb), and 363P2 (160 kb). Cells were streaked on Luria-Bertani (LB) agar plates supplemented with the appropriate antibiotic. A single colony was picked up and subjected to colony-PCR analysis with corresponding STS primers described in Cooper et al., 1997, Genomics 41:185-192 to confirm the authenticity of PAC clones. A single positive colony was used to prepare a 5-ml starter culture and then 1-L overnight culture in LB medium. The cells were pelleted by centrifugation and PAC DNA was purified by equilibrium centrifugation in cesium chloride-ethidium bromide gradient (Sambrook, Fritsch, and Maniatis, 1989, Molecular Cloning: A Laboratory Manual, second edition, Cold Spring Harbor Laboratory Press). Purified PAC DNA was brought to 50 mM Tris pH 8.0, 15 mM MgCl₂, and 25% glycerol in a volume of 2 ml and placed in a AERO-MIST nebulizer (CIS-US, Bedford, Mass.). The nebulizer was attached to a nitrogen gas source and the DNA was randomly sheared at 10 psi for 30 sec. The sheared DNA was ethanol precipitated and resuspended in TE (10 mM Tris, 1 mM EDTA). The ends were made blunt by treatment with Mung Bean Nuclease (Promega, Madison, Wis.) at 30° C. for 30 min, followed by phenol/chloroform extraction, and treatment with T4 DNA polymerase (GIBCO/BRL, Gaithersburg, Md.) in multicore buffer (Promega, Madison, Wis.) in the presence of 40 uM dNTPs at 16° C. To facilitate subcloning of the DNA fragments, BstX I adapters (Invitrogen, Carlsbad, Calif.) were ligated to the fragments at 14° C. overnight with T4 DNA ligase (Promega, Madison, Wis.). Adapters and DNA fragments less than 500 bp were removed by column chromatography using a cDNA sizing column (GIBCO/BRL, Gaithersburg, Md.) according to the instructions provided by the manufacturer. Fractions containing DNA greater than 1 kb were pooled and concentrated by ethanol precipitation. The DNA fragments containing BstX I adapters were ligated into the BstX I sites of pSHOT II which was constructed by subcloning the BstX I sites from pcDNA II (Invitrogen, Carlsbad, Calif.) into the BssH II sites of pBlueScript (Stratagene, La Jolla, Calif.). pSHOT 11 was prepared by digestion with BstX I restriction endonuclease and purified by agarose gel electrophoresis. The gel purified vector DNA was extracted from the agarose by following the Prep-A-Gene (BioRad, Richmond, Calif.) protocol. To reduce ligation of the vector to itself, the digested vector was treated with calf intestinal phosphatase (GIBCO/BRL, Gaithersburg, Md. Ligation reactions of the DNA fragments with the cloning vector were transformed into ultra-competent XL-2 Blue cells (Stratagene, La Jolla, Calif.), and plated on LB agar plates supplemented with 100 μg/ml ampicillin. Individual colonies were picked into a 96 well plate containing 100 μg/well of LB broth supplemented with ampicillin and grown overnight at 37° C. Approximately 25 μl of 80% sterile glycerol was added to each well and the cultures stored at −80° C.

Preparation of Plasmid DNA

Glycerol stocks were used to inoculate 5 ml of LB broth supplemented with 100 μg/ml ampicillin either manually or by using a Tecan Genesis RSP 150 robot (Tecan AG, Hombrechtikon, Switzerland) programmed to inoculate 96 tubes containing 5 ml broth from the 96 wells. The cultures were grown overnight at 37° C. with shaking to provide aeration. Bacterial cells were pelleted by centrifugation, the supernatant decanted, and the cell pellet stored at −20° C. Plasmid DNA was prepared with a QIAGEN Bio Robot 9600 (QIAGEN, Chatsworth, Calif.) according to the Qiawell Ultra protocol. To test the frequency and size of inserts, plasmid DNA was digested with the restriction endonuclease Pvu II. The size of the restriction endonuclease products was examined by agarose gel electrophoresis with the average insert size being 1 to 2 kb.

DNA Sequence Analysis of Shotgun Clones

DNA sequence analysis was performed using the ABI PRISM™ dye terminator cycle sequencing ready reaction kit with AmpliTaq DNA polymerase, FS (Perkin Elmer, Norwalk, Conn.). DNA sequence analysis was performed with M13 forward and reverse primers. Following amplification in a Perkin-Elmer 9600, the extension products were purified and analyzed on an ABI PRISM 377 automated sequencer (Perkin Elmer, Norwalk, Conn.). Approximately 4 sequencing reactions were performed per kb of DNA to be examined (384 sequencing reactions per each of nine PACs).

Assembly of DNA Sequences

Phred/Phrap was used for DNA sequences assembly. This program was developed by Dr. Phil Green and licensed from the University of Washington (Seattle, Wash.). Phred/Phrap consists of the following programs: Phred for base-calling, Phrap for sequence assembly, Crossmatch for sequence comparisons, Consed and Phrapview for visualization of data, Repeatmasker for screening repetitive sequences. Vector and E. coli DNA sequences were identified by Crossmatch and removed from the DNA sequence assembly process. DNA sequence assembly was on a SUN Enterprise 4000 server running a Solaris 2.51 operating system (Sun Microsystems Inc., Mountain View, Calif.) using default Phrap parameters. The sequence assemblies were further analyzed using Consed and Phrapview.

Identification of New Microsatellite Genetic Markers from the Best's Macular Dystrophy Region

Isolation of CA microsatellites from PAC-specific sublibraries, Southern blotting and hybridization of PAC DNA with a (dC-dA)ₙ(dG-dT)ₙ probe (Pharmacia Biotech, Uppsala, Sweden) was used to confirm the presence of CA repeats in nine PAC clones that represent a minimum tiling path. Shotgun PAC-specific sublibraries were constructed from DNA of all 9 PAC clones using a protocol described above. The sublibraries were plated on agar plates, and colonies were transfered to nylon membranes and probed with randomly primed polynucleotide, (dC-dA)ₙ (dG-dT)ₙ, Hybridization was performed overnight in a solution containing 6×SSC, 20 mM sodium phosphate buffer (pH 7.0), 1% bovine serum albumin, and 0.2% sodium dodecyl sulfate at 65° C. Filters were washed four times for 15 min each in 2×SSC and 0.2% SDS at 65° C. CA-positive subclones were identified for all but one PAC clone (527E4). DNA from these subclones was isolated and sequenced as descrobed above for the shotgun library clones.

Identification of simple repeat sequences in assembled DNA sequences. DNA sequence at the final stage of assembly was checked for the presence of microsatellite repeats using a Consed visualization tool of the Phred/Phrap package.

Polymorphism Analysis and Recombination Mapping

Sequence fragments containing CA repeats were analyzed using the PRIMER program; oligonucleotide pairs flanking each of the CA repeats were synthesized. The forward primer was kinase-labeled with [gamma-³²P]-ATP. Amplification of the genomic DNA was peformed in a total volume of 10 μl containing 5 ng/μl of genomic DNA; 10 mM Tris-HCl pH 8.3; 1.5 mM MgCl₂; 50 mMKCl; 0.01% gelatin; 200 μM dNTPs; 0.2 pmol/μl of both primers; 0.025 unit/μl of Taq polymerase. The PCR program consisted of 94° C. for 3 min followed by 30 cycles of 94° C. for 1 min, 55° C. for 2 min, 72° C. for 2 min and a final elongation step at 72° C. for 10 min. Following amplification, samples were mixed with 2 vol of a formamide dye solution and run on a 6% polyacrylamide sequencing gel. Two newly identified markers detected two recombination events in disease chromosomes of individuals from family S1. This limited the minimum genetic region to the interval covered by 6 PAC clones: 519013, 759J12, 756B9, 363M5, 363P2, and 741N15. Identification of the retina-specific EST hit in the pCA759112-2 clone.

A CA-positive subclone (pCA759J12-2) was identified in the shotgun library generated from the PAC 759j 12 DNA by hybridization to the (dC-dA)ₙ (dG-dT)ₙ probe. DNA sequence from pCA759J12-2 was queried against the EST sequences in the GenBank database using the BLAST algorithm (S. F. Altschul, et al., 1990, J. Mol. Biol. 215:403-410). The BLAST analysis identified a high degree of similarity between the DNA sequence obtained from the clone pCA759J12-2 and a retina-specific human EST with GenBank accession number AA318352. BLASTX analysis of EST AA318352 revealed a strong homology of the corresponding protein to a group of C. elegans proteins with unknown function (RFP family). The RFP family is known only from C. elegans genome and EST sequences (e.g., C. elegans C29F4.2 and B0564.3) and is named for the amino acid sequence RFP that is invariant among 15 of the 16 family members; members share a conserved 300-400 amino acid sequence including 25 highly conserved aromatic residues.

A human gene partially represented in pCA759J12-2 and EST AA318352 was dubbed CG1CE (Candidate Gene #1 with the homology to the C. elegans group of genes) and selected for detaled analysis.

BioInformatic Analysis of Assembled DNA Sequences

When the assembled DNA sequences from the nine BMD PACs approached 0.5-1-fold coverage, the DNA contigs were randomly concatenated, and prediction abilities of the program package AceDB were utilized to aid in gene identification.

In addition to the DNA sequence generated from the nine PACs mentioned above, Genbank database entries for PACs 466A11 and 363P2 (GeneBank accession numbers AC003025 and AC003023, respectively) were analyzed with the use of the same AceDB package. PAC clones 466A11 and 363P2 represent parts of the PAC contig across the BMD region (Cooper et al., 1997, Genomics 41:185-192); both clones map to the minimum genetic region containing the BMD gene that was determined by recombination breakpoint analysis in a 12-generation Swedish pedigree (Graff et al., 1997, Hum. Genet. 101: 263-279). Datbase entries for PACs 466A11 and 363P2 represent unordered DNA pieces genereated in Phase 1 High Throughput Genome Sequence Project (HTGS phase 1) by Genome Science and Technology Center, University of Texas Southwestern Medical Center at Dallas.

cDNA Sequence and Exon/Intron Organization of the CG1CE Gene

Genomic DNA sequences from PACs 466A111 and 759J12 were compared with the CG1CE cDNA sequence from EST AA318352 using the program Crossmatch which allowed for a rapid and sensitive detection of the location of exons. The identification of intron/exon boundaries was then accomplished by manually comparing visualized genomic and cDNA sequences by using the AceDB package. This analysis allowed the identification of exons 8, 9, and 10 that are represented in EST AA318352. To increase the accuracy of the analysis, the DNA sequence of EST AA318352 was verified by comparison with genomic sequence obtained from pCA759J12-2, PAC 466A11, and shotgun PAC 759J12 subclones. The verified EST AA318352 sequence was reanalyzed by BLAST; two new EST's (accession numbers AA307119 and AA205892) were found to partially overlap with EST AA318352. They were assembled into a contig using the program Sequencher (Perkin Elmer, Norwalk, Conn.), and a consensus sequence derived from three ESTs (AA318352, AA307119, and AA205892) was re-analyzed by BLAST. BLAST analysis identified a fourth EST belonging to this cluster (accession number AA317489); EST AA317489 was included in the consensus cDNA sequence. The consensus sequence derived from the four ESTs (AA318352, AA307119, AA205892, and AA317489) was compared with genomic sequences obtained from pCA759J12-2, PAC 466A11, and shotgun PAC 759J12 subclones using the programs Crossmatch and AceDB. This analysis verified the sequence and corrected sequencing errors that were found in AA318352, AA307119, AA205892, and AA317489. Comparison of cDNA and genomic sequences revealed a total of 7 exons. The order of the exons from 5′ end to 3′ end was 5′-ex4-ex5-ex6-ex8-ex9-ex10-ex11-3′. BLASTX analysis of the genomic segment located between exons 6 and 8 in PAC 466A11 revealed strong homology of the corresponding protein to a group of C. elegans proteins (RFP family). Since there were no EST hits in the GenBank EST database that covers this stretch of genomic sequence, this part of the CG1CE gene was called exH (Hypothetical ex 7). This finding changed the order of exons in the CG1CE gene to 5′-ex4-ex5-ex6-ex7-ex8-ex9-ex10-ex11-3′. The BLAST analysis of the DNA region located upstream of the exon 4 identified an additional human EST (AA326727) with a high degree of similarity to genomic sequence. Comparison of DNA and genomic sequences revealed the presence of two additional exons (ex1 and ex2) in the CG1CE gene. This finding changed the order of the exons in the CG1CE gene to 5′-ex1-ex2-ex4-ex5-ex6-ex7-ex8-ex9-ex10-ex11-3′. Bioinformatic analysis did not allow the prediction of boudaries between exons 2 and 4, exons 6 and 7, and exons 7 and 8. In addition, there was no overlap between ESTs represented in exons 1 and 2 from one side and exons 4, 5, 6, 7, 8, 9, 10, and 11 from another. There was the possibility of the presence of additional exons in the CG1CE gene that were not represented in the GenBank EST database.

Identification of an Additional Exon and Determination of the Exact Exon/Intron Boundaries within the CG1CE Gene.

To identify additional exon(s) within the CG1CE gene and verify the exonic composition of this gene, forward and reverse PCR primers from all known exons of the CG1CE gene were synthesized and used to PCR amplify CG1CE cDNA fragments from human retina “Marathon-ready” cDNA (Clontech, Palo Alto, Calif.). In these RT-PCR experiments forward primer from ex1 (LF: CTAGTCGCCAGACCTTCTGTG) (SEQ.ID.NO.:9) was paired with a reverse primer from ex4 (GR: CTTGTAGACTGCGGTGCTGA) (SEQ.ID.NO.:10), forward primer from ex4 (GF: GAAAGCAAGGACGAGCAAAG) (SEQ.ID.NO.:11) was paired with a reverse primer from ex6 (ER: AATCCAGTCGTAGGCATACAGG) (SEQ.ID.NO.:12), forward primer from ex6 (EF: ACCTTGCGTACTCAGTGTGGA) (SEQ.ID.NO.:13) was paired with a reverse primer from ex8 (AR: TGTCGACAATCCAGTTGGTCT) (SEQ.ID.NO.:14), forward primer from ex8 (AF: CCCTTTGGAGAGGATGATGA) (SEQ.ID.NO.:15) was paired with a reverse primer from ex10 (CR: CTCTGGCATATCCGTCAGGT) (SEQ.ID.NO.:16), forward primer from ex10 (CF: CTTCAAGTCTGCCCCACTGT) (SEQ.ID.NO.:17) was paired with a reverse primer from ex11 (DR: GCATCCCCATTAGGAAGCAG) (SEQ.ID.NO.:18).

A 50 μl PCR reaction was performed using the Taq Gold DNA polymerase (Perkin Elmer, Norwalk, Conn.) in the reaction buffer supplied by the manufacturer with the addition of dNTPs, primers, and approximately 0.5 ng of human retina cDNA. PCR products were electrophoresed on a 2% agarose gel and DNA bands were excised, purified and subjected to sequence analysis with the same primers that were used for PCR amplification. The assembly of the DNA sequence results of these PCR products revealed that:

(i) exons 1 and 2 from one side and exons 4, 5, 6, 7, 8, 9, 10, and 11 indeed represent fragments of the same gene

(ii) an additional exon is present between exons 2 and 4 (named ex3)

(iii) exon 7 (Hypothetical) predicted by the BLASTX analysis is present in the CG1CE cDNA fragment amplified by EF/AR primers.

Comparison of the DNA sequences obtained from RT-PCR fragments with genomic sequences obtained from pCA759J12-2, PAC 466A11, and shotgun PAC 759J12 subclones was performed using the programs Crossmatch and AceDB. This analysis confirmed the presence of the exons originally found in five ESTs (AA318352, AA307119, AA205892, AA317489, and AA326727) and identified an additional exon (exon3) in the CG1CE gene. Exact sequence of exon/intron boundaries within the CG1CE gene were determined for all of the exons. The splice signals in all introns conform to publish consensus sequences. The CG1CE gene appears to span at least 16 kb of genomic sequence. It contains a total of 11 exons.

Two Splice Donor Sites for Intron 7.

Two splicing variants of exon 7 were detected upon sequence analysis of RT-PCR products amplified from human retina cDNA with the primer pair EF/AR. Two variants utilize alternative splice donor sites separated from each other by 203 bp. Both splicing sites conform to the published consensus sequence.

Identification of 5′ and 3′ Ends of CG1CE cDNA

RACE is an established protocol for the analysis of cDNA ends. This procedure was performed using the Marathon RACE template from human retina, purchased from Clontech (Palo Alto, Calif.). cDNA primers KR (CTAAGCGGGCATTAGCCACT) (SEQ.ID.NO.:19) and LR (TGGGGTTCCAGGTGGGTCCGAT) (SEQ.ID.NO.:20) in combination with a cDNA adaptor primer AP1 (CCATCCTAATACGACTCACTATAGGGC) (SEQ.ID.NO.:21) were used in 5′RACE. cDNA primer DF (GGATGAAGCACATTCCTAACCTGCTTC) (SEQ.ID.NO.:22) in combination with a cDNA adaptor primer AP1 (CCATCCTAATACGACTCACTATAGGGC) (SEQ.ID.NO.:21) was used in 3′RACE. Products obtained from these PCR amplifications were analyzed on 2% agarose gels. Excised fragments from the gels were purified using Qiagen QIAquick spin columns and sequenced using ABI dye-terminator sequencing kits. The products were analyzed on ABI 377 sequencers according to standard protocols.

### EXAMPLE 2

Best's Macular Dystrophy is Associated with Mutations in an Evolutionarily Conserved Region of CG1CE

Genomic DNA from BMD patients from two Swedish pedigrees having Best's macular dystrophy (families S1 and SL76) was amplified by PCR using the following primer pair:

| | | |
|---|---|---|
| exG_left | AAAGCTGGAGGAGCCGAG | (SEQ.ID.NO.:23) |
| exG₁₃ right | CTCCACCCATCTTCCGTTC | (SEQ.ID.NO.:24) |

This primer pair amplifies a genomic fragment that is 412 bp long and contains exon4 and adjacent intronic regions.

The patients were:

Family S1:

S1-3, a normal individual, i.e., not having BMD; sister of S1-4

S1-4, an individual heterozygous for BMD; and

S1-5, an individual homozygous for BMD.

Patients S1-4 and S1-5 had the clinical symptoms of BMD, including morphological changes observable upon ophthalmologic examination.

Family SL76:

SL76-3, an individual heterozygous for BMD; mother of SL76-2

SL76-2, an individual heterozygous for BMD, son of SL-3.

PCR products produced using the primer sets mentioned above were amplified in 50 μl reactions consisting of Perkin-Elmer 10×PCR Buffer, 200 mM dNTP's, 0.5 ul of Taq Gold (Perkin-Elmer Corp., Foster City, Calif.), 50 ng of patient DNA and 0.2 μM of forward and reverse primers. Cycling conditions were as follows:

| | |
|---|---|
| 1. | 94° C. 10 min |
| 2. | 94° C. 30 sec |
| 3. | 72° C. 2 min (decrease this temperature by 1.1° C. per cycle) |
| 4. | 72° C. 2 min |
| 5. | Go to step 2 15 more times |
| 6. | 94° C. 30 sec |
| 7. | 55° C. 2 min |
| 8. | 72° C. 2 min |
| 9. | Go to step 6 24 more times |
| 10. | 72° C. 7 min |
| 11. | 4° C. |

Products obtained from this PCR amplification were analyzed on 2% agarose gels and excised fragments from the gels were purified using Qiagen QIAquick spin columns and sequenced using ABI dye-terminator sequencing kits. The products were analyzed on ABI 377 sequencers according to standard protocols.

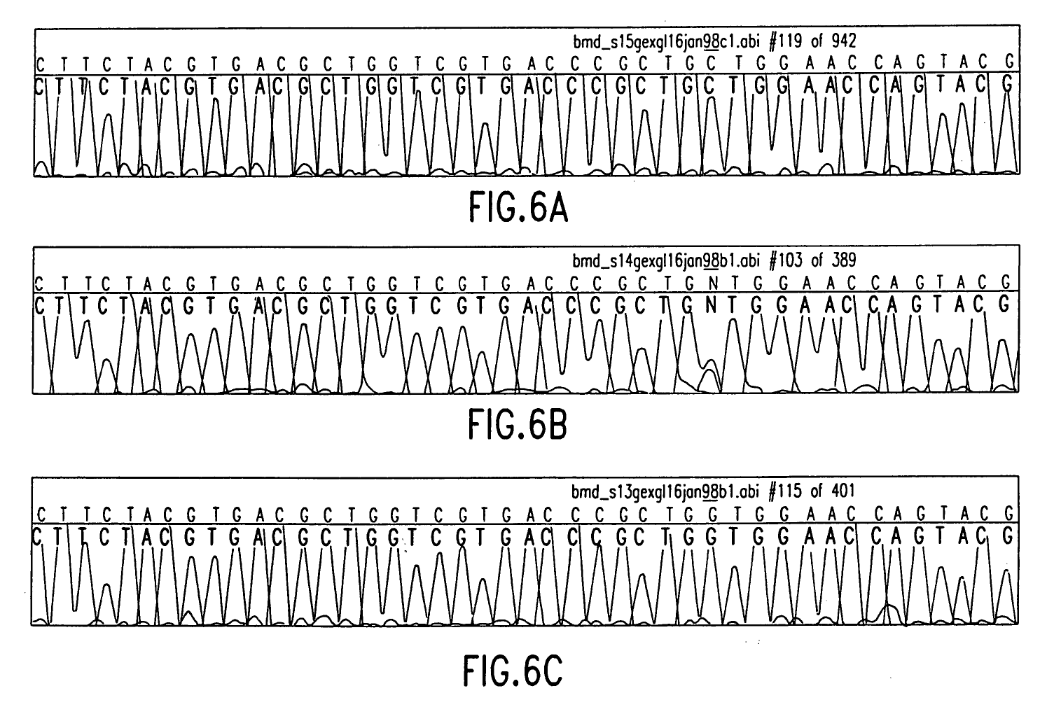

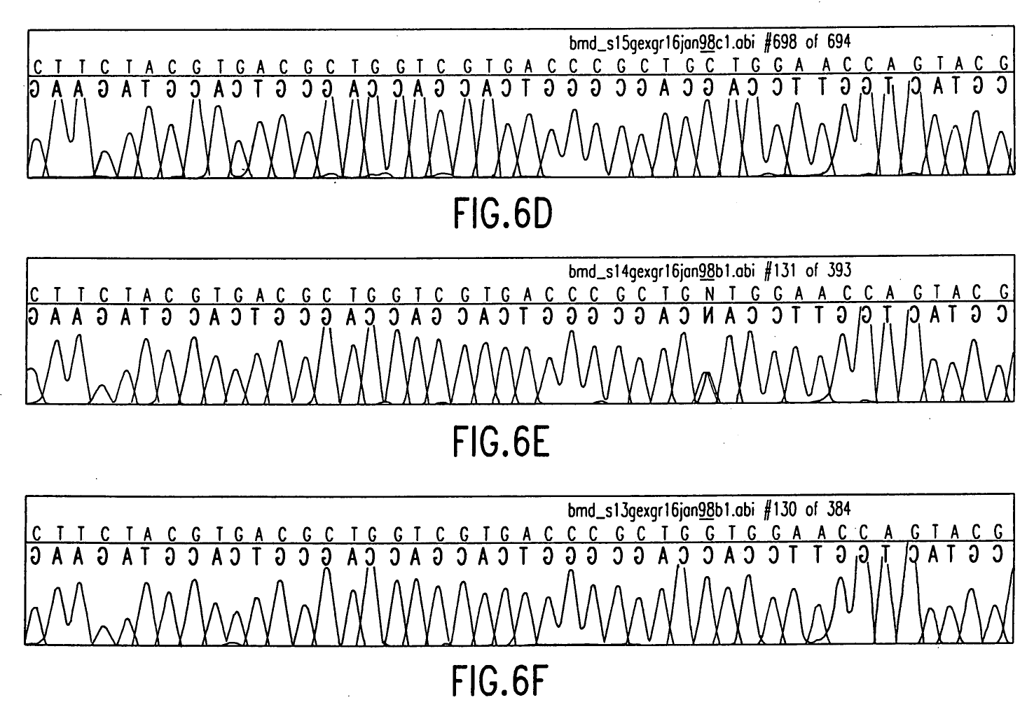

The results are shown in FIG. 6. FIG. 6 shows a chromatogram from sequencing runs on the PCR fragments from patients S1-3, S1-4, and S1-5. The six readings represent sequencing of both strands of the PCR fragments from the patients. As can be seen from FIG. 6, the two patients affected with BMD, patients S1-4 and S1-5, both carry a mutation at position 383 of SEQ.ID.NO.:2. Both copies of the CG1CE gene are mutated in homozygous affected S11-5, while heterozygous affected S1-4 contains both normal and mutated copies of the CG1CE gene. This mutation changes the codon that encodes the amino acid at position 93 of SEQ.ID.NO.:3 from TGG (encoding tryptophan) to TGC (encoding cysteine). Patient S1-3, a normal individual, has the wild-type sequence, TGG, at this codon. This disease mutation that changes this TGG codon to a TGC codon was not found upon sequencing of 50 normal unrelated individulas (100 chromosomes) of North American descent.

Both patients from family SL76 carry a mutation at position 357 of SEQ.ID.NO.:2. This mutation changes the codon that encodes the amino acid at position 85 of SEQ.ID.NO.:3 from TAC (encoding tyrosine) to CAC (encoding histidine). This disease mutation that changes this TAC codon to a CAC codon was not found upon sequencing of 50 normal unrelated individulas (100 chromosomes) of North American descent.

Amino acid positions 85 and 93 of the CG1CE protein are evolutionarily conserved. FIG. 7 demonstrates that position 93 is occupied by tryptophan not only in the CG1CE protein, but also in 15 of 16 related C. elegans proteins. The lone C. elegans protein in which this residue is not tryptophan contains an isofunctional phenylalanine instead. Phenylalanine and tryptophan, both being hydrophobic, aromatic amino acids, are highly similar. Position 85 is occupied by tyrosine and isofunctional phenylalanine in all 16 related C. elgans proteins. Phenylalanine and tyrosine, both being aromatic amino acids, are highly similar.

### EXAMPLE 3

Expression of CG1CE

RT-PCR: RT-PCR experiments were performed on “quick-clone” human cDNA samples available from Clontech, Palo Alto, Calif. cDNA samples from heart, brain, placenta, lung, liver, skeletal muscle, kidney, pancreas, and retina were amplified with primers AF (CCCTTTGGAGAGGATGATGA) (SEQ.ID.NO.:15) and CR (CTCTGGCATATCCGTCAGGT) (SEQ.ID.NO.:16) in the following PCR conditions:

| | |
|---|---|
| 1. | 94° C. 10 min |
| 2. | 94° C. 30 sec |
| 3. | 72° C. 2 min (decrease this temperature by 1.1° C. per cycle) |
| 4. | 72° C. 2 min |
| 5. | Go to step 2 15 more times |
| 6. | 94° C. 30 sec |
| 7. | 55° C. 2 min |
| 8. | 72° C. 2 min |
| 9. | Go to step 6 19 more times |
| 10. | 72° C. 7 min |
| 11. | 4° C. |

The CG1CE Gene was Found to Be Predominantly Expressed in Human Retina and Brain

Northern blot analysis: Northern blots containing poly(A+)-RNA from different human tissues were purchased from Clontech, Palo Alto, Calif. Blot #1 contained human heart, brain placenta, lung, liver, skeletal muscle, kidney, and pancreas poly(A+)-RNA. Blot #2 contained stomach, thyroid, spinal cord, lymph node, trachea, adrenal gland, and bone marrow poly(A+)-RNA.

Primers CF (CTTCAAGTCTGCCCCACTGT) (SEQ.ID.NO.:17) and exC_right (TAGGCTCAGAGCAAGGGAAG) (SEQ.ID.NO.:25) were used to amplify a PCR product from total genomic DNA. This product was purified on an agarose gel, and used as a probe in Northern blot hybridization. The probe was labeled by random priming with the Amersham Rediprime kit (Arlington Heights, Ill.) in the presence of 50-100 μCi of 3000 Ci/mmole [alpha ³²P]dCTP (Dupont/NEN, Boston, Mass.). Unincorporated nucleotides were removed with a ProbeQuant G-50 spin column (Pharmacia/Biotech, Piscataway, N.J.). The radiolabeled probe at a concentration of greater than 1×10⁶ cpm/ml in rapid hybridization buffer (Clontech, Palo Alto, Calif.) was incubated overnight at 65° C. The blots were washed by two 15 min incubations in 2×SSC, 0.1% SDS (prepared from 20×SSC and 20% SDS stock solutions, Fisher, Pittsburgh, Pa.) at room temperature, followed by two 15 min incubations in 1×SSC, 0.1% SDS at room temperature, and two 30 min incubations in 0.1×SSC, 0.1% SDS at 60° C. Autoradiography of the blots was done to visualize the bands that specifically hybridized to the radiolabeled probe.

The probe hybridized to an mRNA transcript that is uniquely expressed in brain and spinal cord.

Mouse probe for the murine ortholog of the GC1CE gene was generated based on the sequence of an EST with GenBank accession number AA497726. The 246 bp probe was amplified from mouse heart cDNA (Clontech, Palo Alto, Calif.) using the primers mouseCG1CE_L (ACACAACACATTCTGGGTGC) (SEQ.ID.NO.:26) and mouseCG1CE_R (TTCAGAAACTGCTTCCCGAT) (SEQ.ID.NO.:27). Due to an extremely low expression level of the CG1CE gene in mouse heart, repetitive amplification steps were used to generate this probe. The authenticity of this probe was verified by sequence analysis of the gel purified DNA band. Northern blot containing poly(A+)-RNA from several rat tissues (heart, brain, spleen, lung, liver, skeletal muscle, kidney, testis) was purchase from Clontech, Palo Alto, Calif. The probe hybridized to an mRNA transcript that is expressed in testis only.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

1. (canceled)

2. (canceled)

3. (canceled)

4. (canceled)

5. (canceled)

6. (canceled)

7. A CG1CE protein, substantially free from other proteins, having an amino acid sequence selected from the group consisting of SEQ ID NO.: 3, SEQ ID NO.:5, and SEQ ID NO.: 29.

8. The CG1CE protein of claim 8 containing a single amino acid substitution.

9. The CG1CE protein of claim 9 where the substitution occurs at position 6, 85, 93, 227, or 299.

10. (canceled)

11. (canceled)

12. The CG1CE protein of claim 8 containing an amino acid substitution where the substitution does not occur in a position where the amino acid present in CG1CE is also present in the corresponding position in one of the C. elegans proteins whose partial amino acid sequence is shown in FIG. 7.

13. An antibody that binds specifically to a CG1CE protein where the CG1CE protein has the amino acid sequence selected from the group consisting of SEQ ID NO.:3 and SEQ ID NO.:5.

14. A method of diagnosing whether a patient carries a mutation in the CG1CE gene that comprises:
(a) providing a DNA sample from the patient;
(b) providing a set of PCR primers based upon SEQ ID NO.:2 or SEQ ID NO.:4;
(c) performing PCR on the DNA sample to produce a PCR fragment from the patient;
(d) determining the nucleotide sequence of the PCR fragment from the patient;
(e) comparing the nucleotide sequence of the PCR fragment from the patient with the nucleotide sequence of SEQ ID NO.:2 or SEQ ID NO.:4;
where a difference between the nucleotide sequence of the PCR fragment from the patient with the nucleotide sequence of SEQ ID NO.:2 or SEQ ID NO.:4 indicates that the patient carries a mutation in the CG1CE gene.

15. The method of claim 15 where the DNA sample is genomic DNA.

16. The method of claim 15 where the DNA sample is cDNA.

17. (canceled)

18. A method for determining whether a substance is an activator or an inhibitor of a CG1CE protein or a mutant CG1CE protein comprising:
(a) recombinantly expressing CG1CE protein or mutant CG1CE protein in a host cell;
(b) measuring the biological activity of CG1CE protein or mutant CG1CE protein in the presence and in the absence of a substance suspected of being an activator or an inhibitor of CG1CE protein or mutant CG1CE protein;
where a change in the biological activity of the CG1CE protein or the mutant CG1CE protein in the presence as compared to the absence of the substance indicates that the substance is an activator or an inhibitor of CG1CE protein or mutant CG1CE protein.
